(19) United States
(12) Patent Application Publication
(10) Pub. No.: US 2026/0188456 A1
(43) Pub. Date: Jul. 2, 2026

(54) MACHINE LEARNING FOR INFUSION PUMPS

(71) Applicant: Fresenius Kabi USA, LLC, Lake Zurich, IL (US)

(72) Inventors: Benjamin G. Powers, Portsmouth, NH (US); Stephen C. Anthony, North Andover, MA (US); George W. Gray, North Andover, MA (US)

(21) Appl. No.: 19/129,666

(22) PCT Filed: Nov. 15, 2023
(86) PCT No.: PCT/US2023/079886

§ 371 Date: May 13, 2025

Related U.S. Application Data
(60) Provisional application No. 63/425,827, filed on Nov. 16, 2022.

(51) Int. Cl. G16H 20/17

(52) U.S. Cl. CPC G16H 20/17

(57) ABSTRACT

Machine learned neural networks or machine learning algorithms may be used in a variety of healthcare methods and systems, such as those relating to drug delivery, drug therapies, and/or infusion pumps. The machine-learned neural networks may receive and process data to provide one or more outputs, such as recommended dose limit settings, potential adverse events, medication misuses, occlusion occurrences, extravasations and/or disconnections, pump failures, and maintenance needs. The machine learning algorithms may be trained on a corpus of relevant data to identify recommended dose limit settings, potential adverse events, medication misuses, occlusion occurrences, extravasations and/or disconnections, pump failures, and maintenance needs.

### BACKGROUND

The present application relates to the use of machine learning to improve infusion pump features.

Infusion pumps are used to administer drugs and other medicaments to patients, typically in a clinical setting. An infusion pump provides a controlled amount of the medicament over time to the patient. The amount is administered pursuant to parameters entered by a clinician into the pump using a pump user interface.

Some infusion pumps use dose error reduction systems to control the settings that are available to a clinician. Some infusion pumps deliver controlled medications such as narcotics which are to be handled according to pre-established protocols. Patient physiological data can inform drug delivery parameters that are patient-appropriate. Some infusion pumps can determine whether an occlusion is present in a delivery line.

Infusion pumps can also be prone to manufacturing defects, maintenance needs, and faults in the field.

### SUMMARY

According to an aspect of the present disclosure According to an aspect of the present disclosure, a computer system includes a data aggregator configured to receive infusion pump programming data including one or more of dose limit, dose, dose rate, rate, volume, duration, administration site and diagnosis from medical infusion pumps in use at a plurality of different care facilities, and a machine-learned neural network configured to receive the infusion pump programming data and to process the infusion pump programming data to identify a set of recommended dose limit settings. The system further includes a storage unit configured to store the set of recommended dose limit settings, and a reporting unit configured to transmit the set of recommended dose limit settings in response to a received request.

According to another aspect of the present disclosure, a method of identifying a potential adverse event associated with drug delivery parameters based on patient physiological data includes receiving patient physiological data, receiving a drug delivery parameter for a proposed drug therapy to be administered to the patient, and processing the patient physiological data and the drug delivery parameter with a machine learned neural network, wherein the machine learned neural network identifies a potential adverse event associated with delivery of the proposed drug therapy to the patient. The method further includes generating an alert message based on the identified potential adverse event, transmitting the alert message to an infusion pump, and generating an audible and/or visual alert at the infusion pump to alert an operator to the existence of a potential adverse event.

According to a further aspect of the present disclosure, a method of using machine learning to avoid adverse events when administering a drug therapy to a patient includes collecting a corpus of data including potential adverse events associated with patient physiological data and drug delivery parameters, and training a machine learning algorithm using the corpus of data, the machine learning algorithm being trained to identify a possible adverse event should a proposed drug therapy be administered to a patient having certain patient physiological data. The method further includes using the machine learning algorithm to receive a proposed drug therapy and patient physiological data and to identify the possible adverse event, and providing an indication of the possible adverse event to a display device.

According to a still further aspect of the present disclosure, a method of using machine learning to identify misuse of controlled medications includes collecting a corpus of data including first infusion data and misuse data indicating misuse of a controlled medication, training a machine learning algorithm using the corpus of data, the machine learning algorithm being trained to identify a possible misuse event based on second infusion pump data, using the machine learning algorithm to receive the second infusion pump data to identify the possible misuse event, and providing an indication of the possible misuse event to a display device.

According to yet another aspect of the present disclosure, a computer system includes a data aggregator configured to receive infusion data from medical infusion pumps in use to dispense controlled medications, and a machine-learned neural network configured to receive the infusion data and to identify potential misuse of one of the controlled medications. The system further includes a storage unit configured to store identified misuse of the controlled medication, and a reporting unit configured to transmit an alert regarding the identified misuse of the controlled medication to a destination device.

According to a further aspect of the present disclosure, an infusion pump includes a processing circuit configured to drive an actuator to infuse a substance from a source to a patient, the processing circuit configured to store infusion data relating to the infusion, a machine-learned neural network configured to receive the infusion data as input data and to process the infusion data to predict an occurrence of an occlusion in the line, and an output device configured to generate an audible and/or visible alert in response to the predicted occurrence of an occlusion in the line.

According to a still further aspect of the present disclosure, a method of using machine learning to predict an occurrence of an occlusion in a line of a delivery set driven by an infusion pump includes collecting a corpus of data from a plurality of infusion pumps, wherein the corpus of data includes pressure in delivery lines used by the infusion pumps over time for a plurality of infusion conditions including normal operation and an occlusion condition, and training a machine learning algorithm using the corpus of data, the machine learning algorithm being trained to predict an occurrence of an occlusion in a line. The method further includes using the machine learning algorithm to receive real time pressure data from an operating infusion pump and to predict an occurrence of an occlusion in the operating infusion pump, and providing an indication of the occlusion to a display on the operating infusion pump.

According to yet another aspect of the present disclosure, an infusion pump includes a processing circuit configured to drive an actuator to infuse a substance to a patient, the processing circuit configured to store infusion data relating to the infusion, and a machine-learned neural network configured to receive the infusion data as input data and to process the infusion data to predict an occurrence of extravasation and/or disconnection of tubing from a patient.

The pump further includes an output device configured to generate an audible and/or visible alert in response to the predicted occurrence of extravasation and/or disconnection of tubing from a patient.

According to a further aspect of the present disclosure, a method of using machine learning to predict an occurrence of extravasation and/or disconnection of tubing from a patient receiving therapy from an infusion pump includes collecting a corpus of data from a plurality of infusion pumps, wherein the corpus of data includes pressure in delivery lines used by the infusion pumps over time for a plurality of infusion conditions including normal operation and extravasation and/or disconnection of tubing from a patient, and training a machine learning algorithm using the corpus of data, the machine learning algorithm being trained to predict an occurrence of extravasation and/or disconnection of tubing from a patient. The method further includes using the machine learning algorithm to receive real time pressure data from an operating infusion pump and to predict an occurrence of extravasation and/or disconnection of tubing from a patient, and providing an indication of the extravasation and/or disconnection of tubing from a patient to a display on the operating infusion pump.

According to a still further aspect of the present disclosure, a method of using machine learning to diagnose a cause of an infusion pump failure in a healthcare setting includes collecting a corpus of data including causes of infusion pump failure and associated operational data from infusion pumps, and training a machine learning algorithm using the corpus of data, the machine learning algorithm being trained to identify a possible cause of an infusion pump failure. The method further includes using the machine learning algorithm to receive operational data from an infusion pump that has experienced a failure in the healthcare setting and to output a predicted cause of the infusion pump failure, and providing an indication of the predicted cause of the infusion pump failure to a display device.

According to yet another aspect of the present disclosure, a computer system for predicting a cause of a failure in an infusion pump includes a data input device configured to receive operational data for the infusion pump, a processing circuit configured to retrieve the operational data using the data input device and to store the operational data in a memory device, and a machine-learned neural network configured to receive the operational data as input data and to process the operational data to predict a cause of a failure in the infusion pump. The system further includes an output device configured to generate an indication of the cause of the failure of the infusion pump for display on a display device.

According to a further aspect of the present disclosure, an infusion pump includes a processing circuit configured to drive an actuator to infuse a substance to a patient, the processing circuit configured to store infusion data relating to the infusion, and a machine-learned neural network configured to receive the infusion data as input data and to process the infusion data to predict a need for a maintenance service for the infusion pump. The system further includes an output device configured to generate an audible and/or visible alert in response to the predicted need for a maintenance service for the infusion pump.

According to a still further aspect of the present disclosure, a method of using machine learning to predict a maintenance need of an infusion pump includes collecting a corpus of data including maintenance tasks of infusion pumps and associated log file data from the infusion pumps, training a machine learning algorithm using the corpus of data, the machine learning algorithm being trained to predict a maintenance need of an infusion pump, and using the machine learning algorithm to receive a log file from an infusion pump and to output a maintenance need of the infusion pump. The method further includes providing an indication of the maintenance need of the infusion pump to a display device.

### BRIEF DESCRIPTION OF THE DRAWINGS

FIG. 1 is a flow diagram of a system for collecting infusion data from a plurality of infusion pumps at a server computer, according to an illustrative embodiment;

FIG. 2 is an illustration of exemplary hard and soft limits and their override or reprogramming, according to an illustrative embodiment;

FIG. 3 is a block diagram of a system for using machine learning for generating recommended dose limit settings for infusion pumps, according to an illustrative embodiment;

FIG. 4 is a display screen generated by the system of FIG. 3, according to an illustrative embodiment;

FIG. 5 is flowchart for systems and methods of using machine learning to identify risk of drug delivery parameters based on patient physiological data, according to an illustrative embodiment;

FIG. 6 is a flowchart for a system and method of using a machine learned neural network to recommend modification of an infusion parameter, according to an illustrative embodiment;

FIG. 7 is a block diagram of an infusion pump having a neural network, according to an illustrative embodiment;

FIG. 8 is a flowchart for a system and method of training and using a neural network to indicate occlusion in an infusion pump, according to an illustrative embodiment;

FIG. 9 is a flowchart for a system and method of training and using a neural network to indicate extravasation and/or a disconnection event, according to an illustrative embodiment;

FIG. 10 is a block diagram of a computer system using a neural network to assist in diagnosing a fault of an infusion pump, according to an illustrative embodiment;

FIG. 11 is a flowchart for a system and method of training and using a machine learning algorithm to predict a cause of failure of an infusion pump, according to an illustrative embodiment; and

FIG. 12 is a flowchart of a system and method of training and using a machine learned algorithm to indicate a maintenance need of an infusion pump, according to an illustrative embodiment.

### DETAILED DESCRIPTION OF ILLUSTRATIVE EMBODIMENTS

In some embodiments, machine learning may be used. Machine learning is a type of artificial intelligence which uses sample data or training data or a training corpus to build a model. The model operates to make predictions or decisions. A machine learning model may use one or more algorithms such as a convolutional neural network, Bayesian networks, nearest neighbor, reinforcement learning, decision tree, federated learning, other algorithms for classification and/or regression, etc. One or more of the algorithms may be open-source algorithms.

In some embodiments, machine learning may comprise an algorithm or application that provides computer systems the ability to perform tasks by making inferences based on patterns found in an analysis of training data. Machine learning may comprise algorithms or other tools that may learn from training data and make predictions about new data. Machine learning algorithms may be configured to build one or more machine learning models or modules from training data configured to receive and analyze an available dataset and make data-driven predictions, decisions, likelihoods or diagnoses expressed as outputs or assessments.

Machine learning may be supervised or unsupervised. Supervised learning can be based on labelled or highlighted aspects or features of training data. Unsupervised learning may rely on automatically finding patterns in training data without requiring labelled or highlighted aspects or features. Machine learning may further be based on reinforcement learning and/or self-learning.

Training data may be selected or curated for the specific machine learning purpose.

A machine learning algorithm may comprise one or more weights which may be configurable by a technician.

Machine learning may further comprise deep learning (e.g., hierarchical learning, deep neural learning, deep structured learning, deep belief networks, recurrent neural networks, convolutional neural networks, etc.), which may comprise using a neural network algorithm that is many layers deep. A first layer may learn simple aspects of the input data and as the layers get deeper, they recognize more complex features of the input data. A final layer is then able to distinguish if there is a condition present in the input data. Deep learning may comprise using at least five layers, at least fifteen layers, at least thirty layers, etc.

In some embodiments, a plurality of different machine learning algorithms may be combined to perform the machine learning function, for example by concatenation, interweaving, input data processing, etc.

In some embodiments, a method may comprise training a machine learning model based on training data and applying the machine learning model to a set of input data to generate a set of output data. The machine learning model may comprise a machine learning probability prediction model.

In some embodiments, a computer system, computing system, server computer, or other processing circuit may be configured to or programmed to operate one or more modules to perform the functions described herein. For example, the computer system may be programmed to operate a data aggregator configured to receive, filter and/or label training data from one or more sources, such as infusion pumps, literature databases, electronic medical records databases or other healthcare computing systems, data files such as spreadsheets or word processing documents, or other data sources.

In some embodiments, a neural network may comprise a framework of machine learning algorithms that work together to classify inputs based on a previous training process. A computer-implement method of training a neural network may comprise collection a set of training data from a database, creating a training set comprising the collected set of training data, a modified set of training data, and/or other data, and training the neural network using the created training set.

In some embodiments, a rules module may be programmed with predetermined rules or other relationships among data. The rules module may comprise rules created by a technician based on research or experimentation (e.g., human-determined algorithms), or the rules may be generated or computed by a machine learning algorithm based on training data comprising at least one known outcome, and optionally without manually prescribing a particular formula or set of rules.

In some embodiments, a machine learned neural network may be trained using training data until a desired performance level is reached. The machine learned neural network may then be deployed in a computing device in a medical environment, such as an infusion pump in a hospital. The machine learned neural network may be configured to receive real time data or other data from current infusions and make determinations based on that data and the trained model of the machine learned neural network. The outputs of the trained model may comprise alerts, adjustment to infusion delivery, notifications, recordation in a patient medical record, or even further learning (e.g., feedback) or training of the model. Thus, in some embodiments, an infusion pump or other medical device may be configured to continue training the deployed neural network as the pump is in use in a clinical setting. A feedback loop may be used to tune weights or other components of the machine learning neural network, to adjust sensitivity thresholds for alerts, etc.

In some embodiments, a machine learning module may be configured to receive training data and to construct a statistical model using one or more classification algorithms. The statistical model may then be used in real time in a medical device to make determinations.

One or more of the computing components, units, modules, aggregators, etc. described herein may be implemented with a cloud server or network which may comprise one or more server computers acting singly or in concert, which may comprise shared resources and o-demand access vis the internet, the resources configured to operate one or more of applications, servers (physical servers, virtual servers, etc.), data storage, development tools, networking capability, etc. Cloud computing may be hosted at a remote data center managed by a cloud services provider. Alternatively, non-cloud computing resources may be used.

One or more of the computing components, units, modules, aggregators, etc. described herein may comprise a processing circuit or control circuit comprising analog and/or digital electronic components, such as microprocessors, microcontrollers, memory devices, application-specific integrated circuits, programmable logic, or other electronic configured to perform the functions described herein by way of hardware programming, software programing, firmware, etc. The features may be embodiment on a tangible and non-transitory computer-readable memory device such as magnetic storage, solid state electronic memory, or other memory devices.

The following features may be implemented using any one or more of the components, algorithms, features, or functions described hereinabove, in various combinations, sequences, orders, or other configurations. Each of the features described below are understood to incorporate the teachings hereinabove into various embodiments to be claimed. Further, aspects of the features described below may be incorporated with aspects of other features described below to realize further embodiments to be claimed.

To avoid errors in drug administration, some infusion pumps hold a library of drug names and associated limits or constraints. For example, a drug may have a hard upper limit for a parameter such as rate of infusion. The hard upper limit is predetermined by a pharmacist or other clinician familiar with the drug. When a user selects the drug and programs a rate of infusion, the infusion pump prevents the pump from being programmed to administer the drug above the hard upper limit. In another example, a drug may have a soft upper limit. When a user selects the drug and attempts to program the rate of infusion above the soft upper limit, an alert is given on the user interface to notify the user they are requesting a parameter value or parameter setting beyond the soft upper limit and asking the user to confirm the rate of infusion. This library may be created—and updated—by a pharmacist, medication safety committee, and/or other clinician.

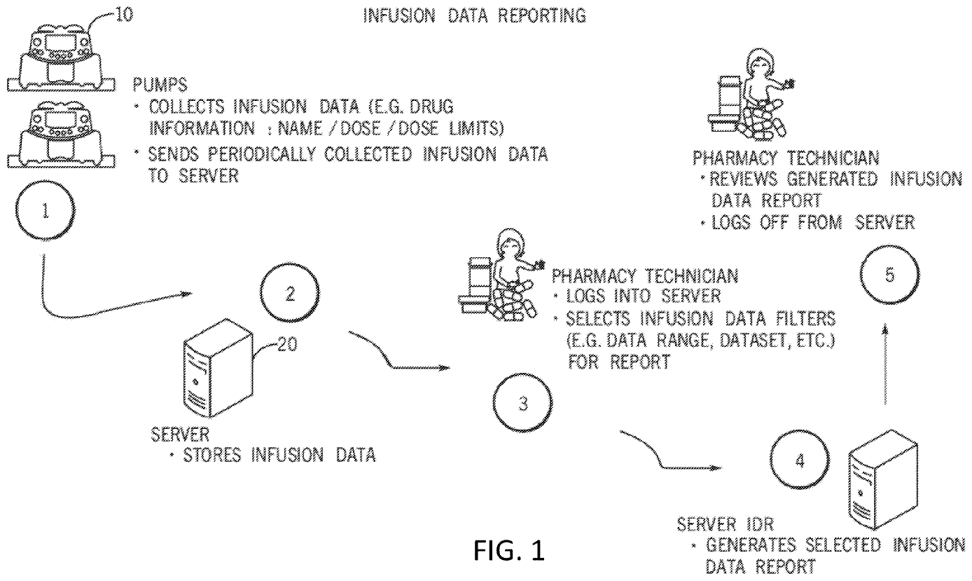

Referring to FIG. 1, a flow diagram of a system for collecting infusion data from a plurality of infusion pumps at a server computer will be described. Infusion pump 10 may be any of a variety of infusion pumps, such as a volumetric infusion pump, a patient-controlled analgesia (PCA) pump, an elastomeric pump, a syringe pump, an enteral or parenteral feeding pump, an insulin pump, etc. At Step 1 in FIG. 1, infusion pump 10 is configured to collect infusion pump history data, such as user key presses on a user interface thereof, alarm data, etc. History data can include drug or infusate name, dose, dose changes, start volumes, rates, stop time, alarm or alert information indicating a cross beyond hard or soft upper or lower limits, etc. Alert data may include an indication that an alert was generated by the pump, a start time for the alert, a care area in which the pump was used during the alert, a drug name of a drug being administered during the alert, time to alert resolution, etc.

At Step 2 in FIG. 1, infusion pump 10 may be configured for wired and/or wireless communication with a server computer 20. Each of pump 10 and server computer 20 may comprise a network interface circuit configured for network communications, such as a Wi-Fi circuit, Bluetooth circuit, Ethernet card, or other network interface circuit. Pump 10 is configured to transmit and server 20 is configured to receive infusion pump data over the respective network interface circuits. Server 20 is configured to store the infusion data from a plurality of infusion pumps, which may be in different care areas, for analysis, whether automated or by a clinician. Infusion data transmissions may be initiated by infusion pump 10 and may occur periodically, intermittently, occasionally, every few minutes, several times per day, or at other regular or irregular frequencies. Infusion data stored at server 20 may be a subset of pump history data that server 20 receives from pump 10.

At Step 3 in FIG. 1, a person may log into server 20 using a terminal (not shown), which may be a user interface for server 20 or may alternatively be a separate computing device or PC. The user opens an application configured to review infusion pump data. Server 20 may be configured to generate one or more reports based on analysis of the infusion pump history data. Reports may be generated in a prescheduled manner or on-demand based on user inputs to the system. Reports may also be sent automatically, without requiring user input, on a scheduled basis, or in response to certain rules being met (e.g., alert triggered, a certain number of alerts triggered, a certain number of override or reprogram events, etc.). The user may select one or more infusion data filters, such as hospital, data set, profile, drug, device type, infusion mode, time and/or date range, etc.

At Step 4 in FIG. 1, the server computer is configured to generate the selected infusion data report or reports.

At Step 5 in FIG. 1, a user analyzes the report data and may make changes to a data set or library used to program infusion pumps 10. For example, a data set may comprise hard limits and/or soft limits to different pump programming parameters, such as infusion rate, dose, infusion time or duration, etc. The limits of the data set may be different for different drugs and may include a “drug X” data set for a drug not known by the data library. Once changes are made to the data set or library, server 20 may be used to remotely download, update, or otherwise program infusion pumps 10 (e.g., by care area, universally, etc.) with the new data set changed by the pharmacist or other user at Step 4.

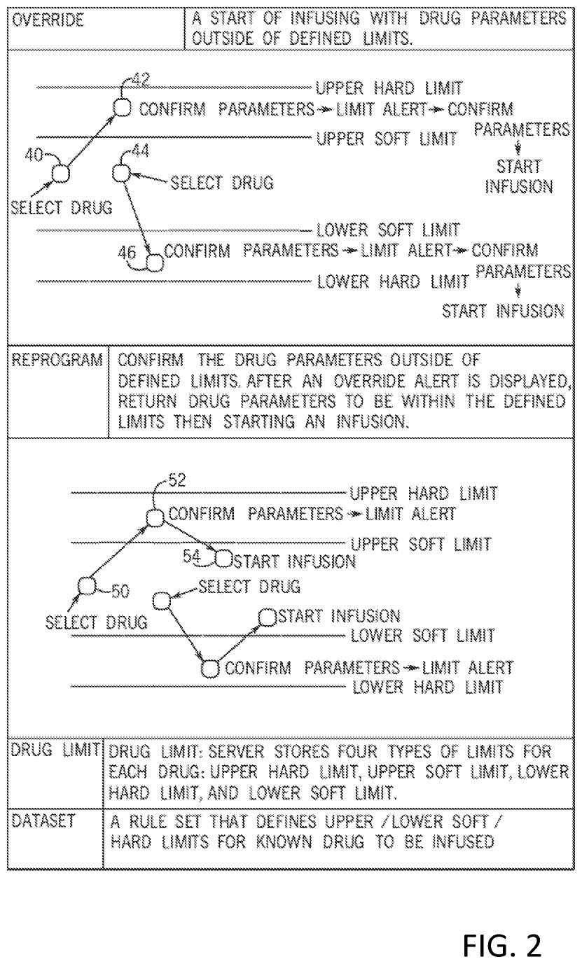

Referring now to FIG. 2, an illustration of pump history data is provided. Each box 40, 42, 44, etc. represents one or more pump history data elements or events which may be independently reported from the infusion pump 10 to the server 20. For example, box 40 represents an indication that a user selected a drug from a list or library of drugs on the infusion pump, which includes the name of the drug selected. Box 40 also represents an initial value of the pump parameter which is within upper and lower limits and which is changeable by a user (e.g., by scrolling up/down, or other input mechanism). Box 42 indicates that the user inputted to the infusion pump a parameter value which was outside of a prestored limit, namely an upper soft limit. Infusion pump 10 then provided an indication that the parameter value was outside the upper soft limit (“LIMIT ALERT”). Infusion pump 10 then received an indication from the user that the input value was confirmed (“CONFIRM PARAMETERS”) and an indication that the infusion was started (“START INFUSION”). Each of these indications can be a separate pump history data element stored in memory of the pump 10 and reported separately to server 20.

A hard limit may refer to a limit beyond which pump 10 does not allow a user to set a value of a parameter. A soft limit may refer to a limit beyond which a pump 10 does allow a user to set a value of parameter, only after the user has been notified with an alert that the value is outside of the soft limit. A pharmacist may program hard and soft limits for different drugs in a drug library in order to guide a nurse, clinician or other user when programming parameters into infusion pump 10. Blocks 40-42 and 44-46 may be referred to as override events, because the history data comprises an indication that a user started an infusion on the pump at the parameter value which was outside of the prestored soft limit, or at the prestored hard limit.

An exemplary reprogram event is also illustrated in FIG. 2. Block 50 represents an infusion pump history data element comprising an indication that a user selected a particular drug from the library. The initial value of the pump parameter may be a default parameter value, for example a parameter value from a previous infusion, a pre-programmed default value from the dataset/library, etc. Block 52 represents an indication that a user inputted to an infusion pump a parameter value which was outside of a prestored limit, namely an upper soft limit. Block 52 also represents an indication that the pump provided a confirmation or alert (“LIMIT ALERT”) to the user and requested confirmation. Block 54 represents an indication that the user returned the infusion pump parameter value to within the prestored limit and the user started the infusion at the parameter value within the prestored limit. A similar illustration is provided for a reprogram event for a lower soft limit. A reprogram event may refer to a confirmation that a drug parameter value is outside of a predefined limit, such as an upper soft limit, that an alert or notification is provided, that the parameter value is returned to be within the predefined limits, and that the infusion is then started.

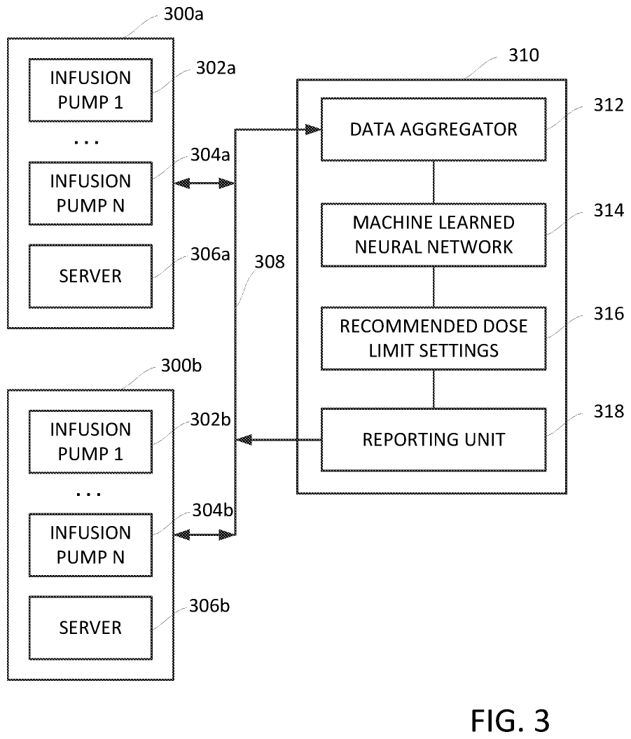

Referring now to FIG. 3, a computer system is shown for using machine learning to identify recommended DERS settings based on aggregated user data, according to an illustrative embodiment. The computer system in FIG. 1 may be configured to communicate over one or more networks 308 with infusion pumps 1-N 302a, 304a at a first participating healthcare institution 300a and with infusion pumps 1-N 302b, 304b at a second participating healthcare institution 300b. The different institutions may be different hospital networks, different corporate entities, different care facilities or other different entities. Each entity may have its own server computer 306a, 306b configured to interface with the infusion pumps for infusion data reporting, software updates, pump monitoring, etc. Server computer 306a, 306b may be configured to transmit certain infusion data over network 308 to a data aggregator 312 of computer system 310. The infusion data may comprise infusion pump programming data, such as dose limit, dose, dose rate, rate or volumetric rate, volume, duration of infusion, administration site and/or patient diagnosis.

The infusion data may comprise dose limit setting data, which itself may comprise any data relating to dose limits. The dose limit setting data may comprise an indication of whether the limit is a hard limit or a soft limit, the value of the limit (e.g. 100 mL/hour), an infusion identifier uniquely identifying an infusion programmed using the limit as a constraint, demographic or other identifying data for the patient undergoing the infusion programmed using the limit as a constraint (e.g., patient population such as neonate, pediatric, adult, geriatric), care practice area associated with the infusion (critical care, med-surg, anesthesia, emergency, etc.), a therapy associated with the infusion (e.g., epidural treatment, patient controlled anesthesia, etc.), an indication of whether the limit was overridden, an indication of whether a parameter limited by the limit was reprogrammed to within the limit, any alerts indicating a limit was exceeded during programming, etc. The infusion data may further comprise other data associated with a drug entity such as default values for concentration and dosing unit.

Data aggregator 312 may be configured to collect the infusion data and store it as training data for a machine learning neural network 314. The training data may be annotated or labeled with one or more of the data elements described above, such as patient population, care practice area, etc. Data aggregation may take place a single time, over a period of months, etc., and may be updated with new training data periodically. Data aggregation may take place after a machine learning neural network is deployed to infusion pumps 302a, b, 304a, b and the infusion pumps are using the neural network during normal operation in the field. In this way, data aggregator 312 may be configured to receive feedback data to further improve the machine learning algorithm. In some embodiments, data aggregator 312 may be configured to receive data from at least 50, at least 100, or at least 1,000 different infusion pumps. In some embodiments, data aggregator 312 may be configured to aggregate data from at least 1,000, at least 5,000, or at least 10,000 infusion events programmed using a DERS library using dose limits.

Machine learned neural network 314 may be configured to receive the infusion pump programming data and to process the infusion pump programming data to identify a set of recommended the infusion pump programming data, such as dose limit settings. For example, if a large number of soft overrides occur for drug X when used in a geriatric care practice, the machine learned neural network 314 may process this data to identify a new soft limit setting which is higher than that used by at least some of the infusion pumps that generated alerts. In another example, the machine learned neural network may be configured to process the data to determine a new default value for a drug which may be different than a default value for the same drug in a different care practice area and/or for a different patient population.

In some embodiments, over time the machine learning neural network may be configured to determine recommended, best, ideal, or “sweet spot” drug parameter settings for the DERS, which may be different for different drugs, drug concentrations, drug therapies, patient populations, and/or care practice areas (e.g., locations such as “5 West” or clinical applications within a healthcare facility).

A storage unit 316 may be configured to configured to store the set of recommended dose limit settings (or other infusion pump programming settings). Storage unit 316 may comprise a server computer or memory device that stores and/or updates recommended dose limit settings or other infusion pump programming settings output from machine learned neural network 314.

A reporting unit 318 may be coupled to the storage unit and configured to transmit a set of the recommended dose limit settings (or other infusion pump programming settings) in response to a received request. For example, the received request may come from an infusion pump 302a, 304a or from a server computer 306a in communication with the infusion pump. The request may be generated automatically or in response to manual user input or request. In one embodiment, a drug library editor module may be provided as an application operating on server computer 306a and/or deployed remotely over network 308 and accessible by server computer 306. The drug library editor module may allow a pharmacy technician or biomedical engineer to log in and create, edit and/or deploy drug libraries for different infusion pumps or groups of infusion pumps (e.g., segmented by care practice area). The drug library editor may be configured to receive user inputs for dose limits and display selected dose limits. The drug library editor may enable a user to import, export and edit whole drug libraries and individual drug library values to control and customize a drug library according to hospital preferences. Upon analyzing the data pool of each hospital, one or more server computers may develop a rules engine for each hospital, wherein the rules engine may be the same or different for each hospital depending on clinical need, preference, and/or risk tolerance.

The drug library editor may provide a screen wherein the set of recommended dose limit settings (or other infusion pump programming settings) from the machine learned neural network are displayed, thereby allowing a user easy access to the recommended settings when constructing or editing a drug library. In this way, the user can benefit from aggregated user data over a plurality or many different medical facilities that has been processed with machine learning to provide recommended dose settings. The recommended dose settings may comprise different settings for the same drug depending on patient population, care practice area, or other characteristics of the set of infusion pumps for which the user is constructing a drug library.

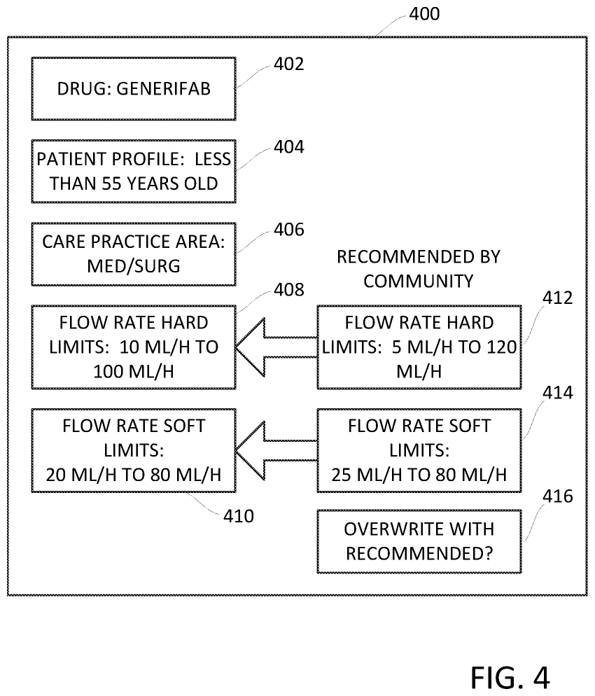

Referring to FIG. 4, an exemplary screen of a drug library editor is shown, according to an illustrative embodiment. The screen comprises a first input field 402 configured to allow a user to select a drug from among a plurality of drugs (e.g., via a drop-down menu, via a search, etc.). A second input filed 404 is configured to allow a user to select a patient profile. A third input field 406 is configured to allow a user to select a care practice area. Input fields 408 and 410 allow a user to view and/or edit hard and soft limits for a flow rate for the drug. According to one advantageous aspect, recommended settings for the flow rate may be displayed at display areas 412 and 414, receiving data from the machine learned neural network 314 and/or the storage unit or reporting unit 318. Arrow indicators may be provided to suggest the option to the user of overwriting/editing the flow rate hard and/or soft limits with the recommended settings. A legend may be provided above the display areas 412, 414 to explain to the user the source of the recommended settings. A user input device 416 may be provided to allow a user to overwrite one or more of the dose limit settings shown in display areas 408, 410 with the recommended settings.

In some embodiments, computer system 310 and/or servers 306a, 306b may further comprise a programming unit configured to program a set of medical infusion pumps based on the set of recommended dose limit settings. The medical infusion pumps may then be configured to administer and to administer medicaments to patients using the updated dose limit settings. For example, a user may use the recommended does limit settings to extend the flow rate beyond 10 mL/hour to 5mL/hour based on a revised hard limit flow rate, as illustrated in the example of FIG. 4.

While the feature of FIGS. 3-4 is described as a computer system comprising a machine-learned neural network, the feature may alternatively be implemented as a method. The method may comprise training the machine learning model with the data collected by data aggregator 312. The method may further comprise performing feature engineering on a training corpus collected by data aggregator 312 to produce a revised training corpus. The method may further comprise using the trained model to generate recommended dose limit settings. The recommended dose limit settings may be generated to reduce overrides, reprograms, and/or other alerts. The recommended dose limit settings may be generated to reduce adverse events associated with administering a drug. Adverse event data may further be received by data aggregator 312 from manual entry and/or automatic entry from medical records databases.

In some embodiments, the method may comprise processing the aggregated dose limit setting data, and optionally other data discussed herein, with a machine learning neural network to determine a class of recommended dose limit settings selected to accomplish one or more business objectives, such as reducing overrides, reducing reprograms, reducing adverse events, etc.

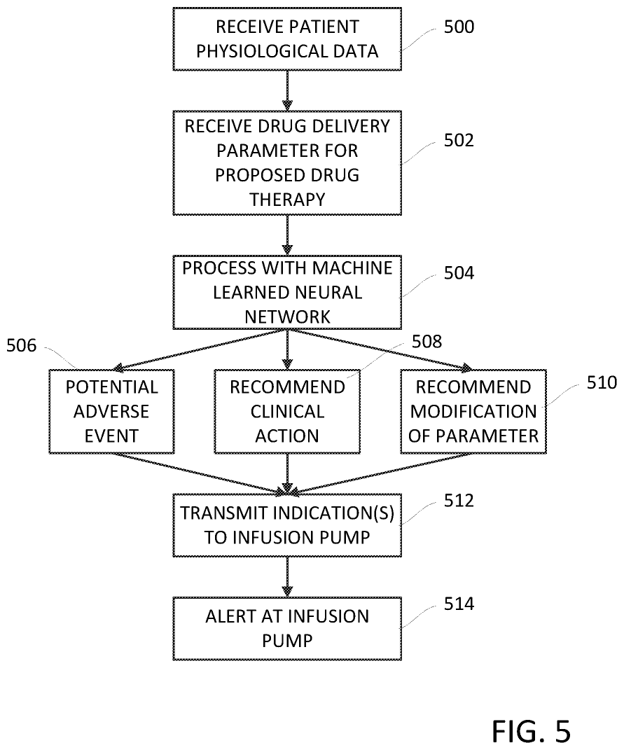

Referring now to FIG. 5, a method of identifying a potential adverse event associated with drug delivery parameters based on patient physiological data will be described, according to an illustrative embodiment. The method may comprise receiving patient physiological data at a block 500. This data may be received at a server computer remote from the infusion pumps, at server computers local 306a, 306b to a healthcare facility, or at other computers. The patient physiological data may comprise one or more patient vital signs, such as blood pulse rate, temperature, respiration rate, blood pressure, etc.). The patient physiological data may comprise clinical laboratory data such as a result of a blood test. The patient physiological data may further comprise a patient diagnosis, patient age (or age range) and patient acuity data (e.g., for one or more attributes, such as physical, psychological, urgency/triage scales, etc.).

At a block 502, the computer may be configured to receive one or more drug delivery parameters for a proposed drug therapy to be administered to the patient. For example, a clinician may program into an infusion pump a drug therapy comprising one or more of drug name or ID, concentration, dose rate, dosage, flow rate, volume to be infused, etc. The clinician may also program on the pump the condition for which the therapy is being administered or the therapeutic effect expected in response to the administered therapy. The infusion pump may transmit one or more of these drug delivery parameters to the computer before the therapy is begun. This method can allow a machine learned neural network, or other rules-based engine, to assess risk of the proposed therapy before the proposed therapy begins.

At a block 504, the method may comprise processing the patient physiological data and the drug delivery parameter with a machine learned neural network. The machine learned neural network may be configured to identify a potential adverse event 506 associated with delivery of the proposed drug therapy to the patient. For example, the neural network may predict that the delivery may cause an inadvertent bleed based on the patient physiological data and drug delivery parameters. Other potential adverse events that may be predicted by the neural network may include an unexpected increase or decrease in heart rate, respiration rate, blood pressure, SpO2 and/or temperature. Other potential adverse events that may be predicted by the neural network may include an unexpected change or lack of change in the patient's ECG or EEG, or other adverse events.

The machine learned neural network may be configured to make other determinations or predictions, such as a recommended clinical action, such as shown at block 508. A recommended clinical action might be to monitor a physiological parameter of the patient during administration of the medication, or other clinical actions. The machine learned neural network may further be configured to recommend a modification of a drug delivery parameter, as shown at block 510. For example, the network may recommend reducing a drug delivery parameter, changing a drug delivery parameter to a particular value, etc. One or more of the assessments, predictions, conclusions, or recommendations of blocks 506, 508 and/or 510 may be implemented in different embodiments.

At a block 512, the method may comprise generating an alert message based on the identified potential adverse event and/or transmitting the alert message to an infusion pump. The alert message may comprise an indication of the potential adverse event (e.g., inadvertent bleeding), a recommended clinical action (e.g., monitor patient's labs), and/or a recommended modification of a delivery parameter (e.g., reduce dosage to X). The alert message may be received by a central computer such as server 306a, 306b for display and/or may be received by the infusion pump that has been programmed with the delivery parameters for the drug to be delivered. At a block 514, the pump may be configured to generate an audible and/or visual alert to alert an operator to the existence of a potential adverse event. Textual and/or graphical messages may accompany the alert with further instructions to the clinician to carry out the recommended actions. The method may further comprise transmitting an indication of one or more of the drug delivery parameters and/or patient physiological data associated with the indication of the potential adverse event for display on the infusion pump or another computer.

According to one example, the machine learning neural network could be trained to indicate potential risks for a patient. The neural network may be configured to receive patient physiological data indicating the patient is a male with a diagnosis of stroke at age 74 with a “high risk” acuity. The drug therapy to be delivered may be heparin. The neural network may output a potential risk of an inadvertent bleed and may recommend clinical lab monitoring for an aPTT if the drug therapy is to be implemented.

According to another example, an infusion pump may generate an alert indicating there is a potential risk of an inadvertent bleed and display the identified parameter(s) that are indicators of that risk (e.g., clinical lab result for aPTT exceeds 91). The infusion pump may display an indication that if the programmed heparin therapy is implemented or continued that an inadvertent bleed may result. The infusion pump may further display a recommendation to decrease the dose to below 500 units/hour.

In another embodiment, a method of using machine learning to avoid adverse events when administering a drug therapy to a patient comprises first collecting a corpus of data comprising potential adverse events associated with patient physiological data and drug delivery parameters. For example, the corpus of data may be created based at least in part on work done by a clinical team researching medical journals to identify risks and how the risks manifest themselves through vital sign and clinical lab measurements. The collection of data can be manual and/or electronic, operated by a computer algorithm. In one example, a computer algorithm may be configured to operate a search engine crawler algorithm to scan web pages and/or research paper databases and add the research papers to a searchable index. The algorithm may then be configured to identify risks associated with the administration of medications via infusion pumps as well as the patient physiological data such as vital signs and clinical lab measurements. The data collected may be used to train a machine learning algorithm to identify the risks based on patient physiological data and drug delivery parameters input later.

In another embodiment, the collection of data may comprise collecting length of stay of a patient and outcome information, either or both of which may be obtained from an electronic medical record. These may be used by a machine learning algorithm to draw correlations between these inputs and a patient falling outside a normal vital sign and/or clinical lab limit. These inputs, in addition to impacting the patient's health, also directly impact the cost of case. There are many ‘quiet’ indicators of poor outcomes but many go undetected or seen as outliers that are associated with a specific patient. For example, unexpected, elevated or decreased heart rates, respiration rates or blood pressures of a patient when administered a drug can be an indication of a larger health condition. When seen across a population, these physiological parameters of a patient may be interpreted by a neural network as more serious if pointed out as a trend across a larger patient population. Certain changes in physiologic parameters that may go undetected by a clinician (for example during a shift change) may result in adverse outcomes and extended lengths of stay. A machine algorithm may be trained with training data comprising physiological parameters or changes thereto that correlate with such adverse outcomes to alert caregivers and lead to better patient outcomes. For example, continuing to administer heparin after a lab result indicating an adverse effect could result in a bleed and other detrimental effects on organs. Thus, a machine learning algorithm may be trained to detect a potential adverse outcome, such as an extended length of stay, in response to an abnormal lab result in the presence of administered heparin.

After the corpus of training data comprising potential adverse events associated with patient physiological data and drug delivery parameters has been collected, the method may further comprise training a machine learning algorithm using the corpus of data. The machine learning algorithm may be trained to identify a possible adverse event should a proposed drug therapy be administered to a patient having certain patient physiological data. The method may further comprise using the machine learning algorithm to receive a proposed drug therapy (e.g., heparin at a specified delivery rate and/or dose) and patient physiological data (e.g., patient age, acuity, diagnosis, vital signs, etc.) and to identify the possible adverse event (e.g., inadvertent bleeding). The method may further comprise providing an indication of the possible adverse event to a display device, such as a display device on the infusion pump that is to be programmed or another display device (e.g., handheld computer or smartphone, laptop computer, etc.). As with the embodiment described with reference to FIG. 4, the method of this embodiment may further comprise providing recommended clinical action(s) and/or recommending a modification of a drug delivery parameter, as well as other steps described in FIG. 5.

The methods described with reference to FIG. 5 may be combined with any of the features described herein to realize new embodiments.

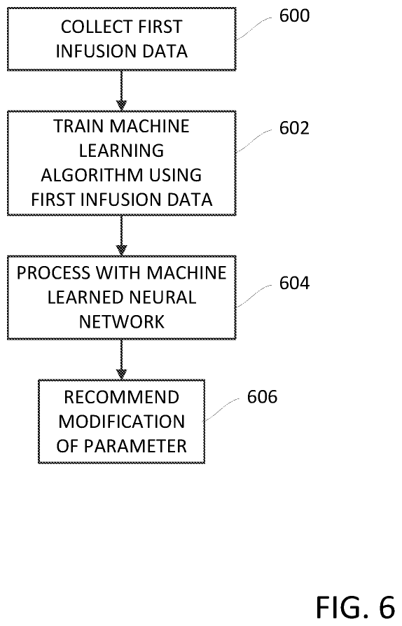

Referring now to FIG. 6, a system and method of using machine learning to track handling of controlled medications will be described, according to illustrative embodiment. Controlled medications such as narcotics are to be handled in accordance with designated procedures to prevent misuse. In one example, a clinician may deliver less than a prescribed dose of a controlled medication to a patient and keep the remainder of the dose for their own use or to sell. FIG. 6 describes a method of using machine learning to identify different types of misuse of controlled medications in different clinical settings. Controlled medications may comprise, for example, medications listed on Schedule II of the U.S. Controlled Substances Act (CSA) such as codeine, oxycodone, hydrocodone, hydromorphone, morphine, opium extracts, fentanyl, methadone, amphetamines, etc. Controlled medications may alternatively be defined as medications on another schedule of the CSA, such as Schedule III, comprising acetaminophen with codeine, ketamine, anabolic steroids, testosterone, etc., or Schedule IV drugs such as certain benzodiazepines, lower doses of codeine, ketamine, anabolic steroids, etc.

At a block 600, the method may comprise collecting a corpus of data comprising first infusion data and misuse data indicating misuse of a controlled medication. The first infusion data may be collected directly from infusion pumps based on infusions performed by the infusion pumps, wherein data may be collected from multiple different healthcare facilities in different geographic locations (e.g., different states, different cities, different health complexes, etc.). Infusion data may comprise a data field indicating whether the medication is a controlled medication or not a controlled medication, whether the medication is a narcotic, and/or a type of controlled medication (e.g., Schedule II or III, etc.). The first infusion data may comprise instances of known misuse associated with the infusion. The first infusion data may be annotated or labelled manually to indicate the presence of a known misuse event associated with the infusion. The first infusion data may come from sources other than infusions, such as from research done on misuse of medications and associated characteristics of infusion data that were present when a controlled medication was being misused.

Misuse events may comprise failure to administer a controlled medication, administering less than a controlled medication, administering more controlled medication than normal for a patient having predetermined characteristics (e.g., age, condition, care practice area., body mass index, etc.), a frequency of administration of controlled medication, underinfusion of controlled medications relative to non-controlled medications.

At a block 602, the method may comprise training a machine learning algorithm using the corpus of data, the machine learning algorithm being trained to identify a possible misuse event based on second infusion pump data. One or more of the algorithms described herein may be used in the training, such as a convolutional neural network, deep learning, Bayesian network, nearest neighbor, reinforcement learning, etc. The training may comprise setting weights or other logic within the machine learning algorithm to generate a machine learned algorithm. The machine learned algorithm may be deployed in the firmware of infusion pumps or may be configured to operate remotely at a server computer based on second infusion pump data being received during clinical use of infusion pumps.

At block 604, the machine learned algorithm may be configured to receive second infusion pump data collected during operating of one or more infusion pumps and to identify possible misuse events. For example, the identified possible misuse event may comprise an underinfusion of controlled medications relative to non-controlled medication by a particular staff member. In another example, the possible misuse event may be indicated based on an infusion pump being programmed with a dose larger than typical for a particular patient followed by an interruption or termination of the infusion before the full dose has been delivered. The machine learning algorithm may be configured to aggregate infusion data and potential misuse events over a number of infusions (e.g., at least 50 infusions, at least 500 infusions, etc.) and identify a misuse event having a certain frequency across infusion pumps and even healthcare facilities.

At block 606, the method may comprise providing an indication of the possible misuse event to a display device. The display device may be associated with a server computer at a location remote from the infusion pump that triggered the potential misuse. The display device may alternatively be a smartphone, tablet, laptop, or other computer associated with a member of leadership of a care practice area and/or a designated hospital monitoring personnel charged with monitoring use of infusion pumps. In one embodiment, the method may comprise transmitting an alert based on the indication of possible misuse as an advisory to a plurality of different healthcare facilities to assist the healthcare facilities in preventing misuse of particular controlled medications and/or by particular types of staff.

In another embodiment, a computer system may comprise a data aggregator configured to receive infusion data from medical infusion pumps in use to dispense controlled medications. The controlled medications may comprise one or more medications on Schedule II of the CSA and/or Schedule III of the CSA, or other controlled medications. For example, a customized list of medications may be used to define controlled medications. The medical infusion pumps may be deployed in use in the same or different healthcare facilities or healthcare entities. Actual infusion data from use on patients may be collected by the data aggregator. A machine-learned neural network may be configured to receive the infusion data and to identify potential misuse of one or more of the controlled medications. The machine-learned neural network may be configured to identify underinfusion of one of the controlled medications and/or underinfusion by a particular clinician or particular clinical role within the facility and/or underinfusion by clinicians within a particular care practice area.

The computer system may comprise a storage unit configured to store the identified misuse of the controlled medication, along with characteristics of the misuse, such as the name of the drug, the type of pump associated with the misuse event, the clinical staff role associated with the misuse, time of day, patient demographic, etc. A reporting unit may be configured to transmit an alert regarding the identified misuse of the controlled medication to a destination device. The reporting unit may be configured to transmit the alert using a notification server to care unit leadership and/or a designated hospital monitoring personnel, for example by electronic mail, text message, encrypted communication, etc.

In one embodiment, the computer system may be configured to receive feedback in response to the alert. The feedback may be used to retrain the machine-learned neural network. An updated machine-learned neural network may be deployed to infusion pumps for subsequent infusions. For example, if the machine learned neural network identifies a potential misuse event which is deemed by a human operator not to be a misuse event, the human operator may annotate that data and use it as training data to further train the neural network to improve the precision of identifying misuse events.

In some embodiments, the machine-learned neural network may be configured to identify a pattern of infusion data as indicating misuse for a first care practice area and to identify the same pattern of infusion data as not indicating misuse for a second care practice area. For example, use of a controlled medication at a high dose may be expected in a med/surg unit for a patient having a large BMI but may be unexpected in a NICU unit for an infant patient.

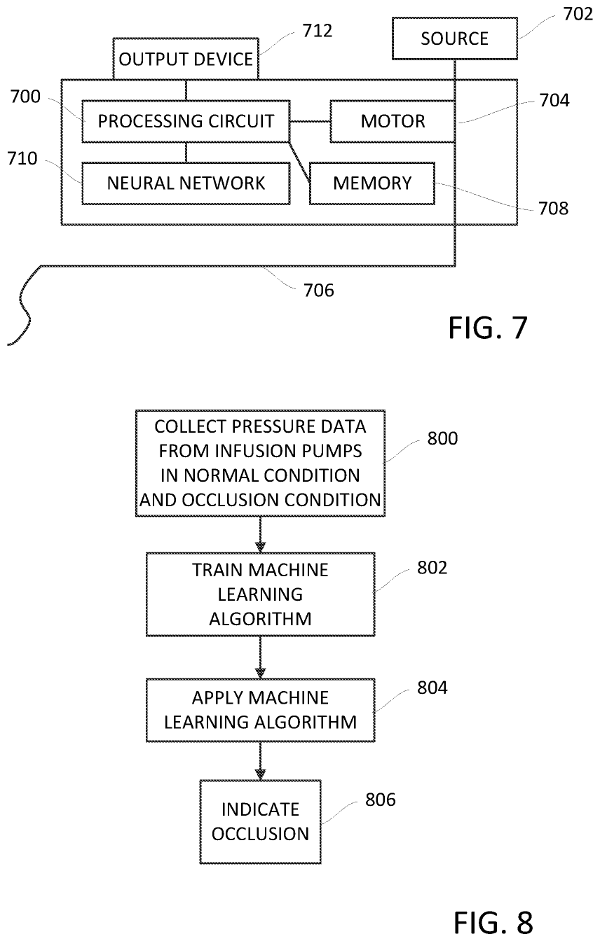

Referring now to FIG. 7, a system and method for using machine learning to predict occlusion in an infusion pump set will be described, according to illustrative embodiments. The infusion pump may be configured with an occlusion detection algorithm that monitors pressure in a line 706 of a delivery set that delivers a substance from a source 702 (e.g., bag, syringe, etc.) to a patient (not shown). The delivery set may be a single-use assembly of tubes or lines, valves, Y-connectors, and/or bags, which may be designed to be disposed of after a single use. The occlusion detection algorithm may be configured to filter, average, count, compare to threshold(s) or otherwise process the pressure data to determine the presence of an occlusion downstream (between pump and patient) or upstream (between source and pump) in infusion line 706. Upon detection, an alarm is triggered (with visual and/or audible alert) to alert a patient or caregiver to remedy the occlusion, such as be removing a kink in the line or replacing the set.

In one embodiment, an infusion pump may comprise a machine-learned neural network 710 configured to receive certain infusion data as input data and to process the infusion data to predict an occurrence of an occlusion in the line. A processing circuit 700 may be configured to drive a motor 704 to infuse a substance from a source 702 through an infusion line 704 to a patient. The processing circuit may be configured to receive, generate, or otherwise store infusion data relating to the infusion, such as pressure in line 706 at one or more locations along the line, characteristic data of the infusion line (e.g., gage or bore size of the line, material of the line, elasticity of the material, etc.), viscosity of the substance being delivered, name of the substance being delivered, and/or other data related to the infusion. The processing circuit 700 may be configured to store one or more of the input data in memory 708. A machine-learned neural network 710 may be configured to receive the data and to use the data to predict an occurrence of an occlusion in the line. In the event of a predicted occurrence of an occlusion, the processing circuit 700 may be configured to trigger an output device 712 to generate an audible and/or visible alert in response to the predicted occurrence of an occlusion in the line.

In one embodiment, the infusion data comprises data from a sensor configured to determine pressure in the infusion line, wherein the machine-learned neural network is configured to process the sensor data to predict the occurrence of an occlusion in the line. The sensor may comprise a motor current sensor or a sensor in direct contact with the line. The sensor may be configured to determine downstream pressure in the line between a pumping actuator and the patient.

In some embodiments, the infusion data may comprise a characteristic of the infusion line and the machine-learned neural network may be is configured to receive the characteristic of the infusion line as input data and to process the characteristic of the infusion line to predict the occurrence of an occlusion in the line.

In some embodiments, infusion data may further comprise a viscosity of the substance being infused and the machine-learned neural network may be configured to receive the viscosity of the substance being infused as input data and to process the viscosity of the substance being infused to predict the occurrence of an occlusion in the line.

Referring now to FIG. 8, a system and method of using machine learning to predict an occurrence of an occlusion in a line of a delivery set driven by an infusion pump will be described, according to an illustrative embodiment. In this embodiment, a machine learning algorithm is trained and then deployed for use. Training, deployment, and/or use may be done by different types of computers in different settings, such as an embedded computer in an infusion pump, a local server computer, a remote server computer, a cloud computing resource, etc. At a block 800, the method may comprise collecting a corpus of data from a plurality of infusion pumps to be used for training the machine learning algorithm. The collection of data may take place over days, weeks or months, or may be done based on data previously collected for other purposes (e.g., infusion data reporting, electronic medical records, continuous improvement, etc.). The corpus of data may be collected from infusion pumps operating at different healthcare facilities by different corporate entities, such as different hospital systems. In this embodiment, the corpus of data may comprise pressure data in delivery lines used by the various infusion pumps (e.g., each pump having its own delivery line). The pressure data may be over time such that a trend or signature of the pressure data may be detected and/or predicted. For example, an increasing pressure in a line over a period of several seconds, several minutes, etc., may indicate the presence of an occlusion. Alternatively, the increase may instead indicate a transient change in pressure to be expected during a normal infusion condition. Thus, the corpus of data may comprise pressure over time data for a plurality of infusion conditions comprising normal operation and an occlusion condition so that the neural network can be trained to distinguish one from another. The data may be manually annotated and/or curated to indicate the condition or the annotation may be done automatically by the infusion pump occlusion detection algorithm upon detection of an occlusion (e.g., by comparing pressure to a threshold, or comparing filtered or averaged pressure to one or more thresholds, etc.). Infusion data representing additional conditions may be collected, such as data associated with a bolus delivery condition (e.g., delivery of a certain quantity at a faster rate than a programmed infusion rate at a later time during a delivery course). Infusion data may also represent a transport condition of the infusion pump, which can be manually input to the pump or automatically determined based on location or navigation sensors (e.g., accelerometers, motion sensors, etc.), to indicate the infusion pump is being transported from one clinical location to another clinical location.

At a block 802, the method may comprise training a machine learning algorithm using the corpus of data, the machine learning algorithm being trained to predict an occurrence of an occlusion in a line. The machine learning algorithm may generate weights or other algorithmic features for processing infusion data received at a future time to make output data in the form of predictions or other determinations relating to occlusions.

At a block 804, the machine-learned neural network or machine learning algorithm may be deployed to infusion pumps (or other computing devices) to receive real time pressure data from an operating infusion pump and to predict an occurrence of an occlusion in the operating infusion pump. The real time pressure data is analyzed in the course of an infusion in process and may be done in the context of a typical use scenario of the infusion pump in a clinical setting. The prediction may be in the form of a simple determination or conclusion based on future input data.

At a block 806, the method comprises providing an indication of the occlusion to a display on the operating infusion pump, so that a clinician or patient may take action to pause the infusion, clear the occlusion, unkink a tubing, etc. The indication may be transmitted to a central monitoring station in a care practice area and/or to handheld devices carried by predetermined clinicians over a suitable wireless network.

In some embodiments, the corpus of data may comprise two or more (or three or more) of drug name, drug viscosity, programmed infusion rate, and administration set type, The machine language algorithm may be trained with the two or more of drug name, drug viscosity, programmed infusion rate, administration set type and/or other environmental drivers.

In some embodiments, the corpus of data may comprise two or more of (or three or more of) patient weight or other patient characteristics or physiological data, infusion route (e.g., PICC (peripherally inserted central catheter) line, CVC (central venous catheter), etc.) and care practice area (e.g., NICU, med/surg, etc.). The machine language algorithm may be trained with the two or more of patient weight, infusion route and care practice area.

In some embodiments, the training data may be collected from a plurality of infusion pumps with an occlusion detection algorithm. The infusion pumps may be recording pressure in delivery lines used by the infusion pumps over time. The training data may be collected into the corpus of data in response to the infusion pump detecting an occlusion condition. For example, once an occlusion alarm is raised, the data set that triggered that alarm may be uploaded into a corpus of training data). The machine learning algorithm may then be trained with the collected data comprising the recorded pressure over time.

In some embodiments, an infusion pump may be configured to prompt a clinician to select from among different conditions being observed so that infusion data associated with the condition may be manually annotated by clinicians in the field. For example, if an occlusion alarm is triggered, the pump may display one or more potential conditions, such as “kinked line,” “occlusion,” “bolus delivery”, “transport condition” and a clinician may make an observation about the condition and select one or more of the displayed options to annotate the infusion data associated with that condition. This annotated infusion data may be transmitted to a server computer for training and/or updating the training (e.g., with feedback) of a machine learned neural network.

In some embodiments, a machine learned neural network may be deployed to an infusion pump. The machine learned neural network may be configured to make a prediction that an occlusion is present or imminent. The infusion pump may, in response, display an alert to a clinician. The clinician may observe the circumstances of the pump and conclude there is no occlusion and that the machine learned neural network was incorrect. The infusion pump may be configured to prompt the clinician with a displayed prompt to confirm or deny the presence of the occlusion. The infusion pump may be configured to receive an indication that no occlusion is present from the operator. The infusion pump may annotate a set of infusion data with this indication. The annotated infusion data can be used to retrain or update training (via feedback) of the machine learned neural network to improve the learning.

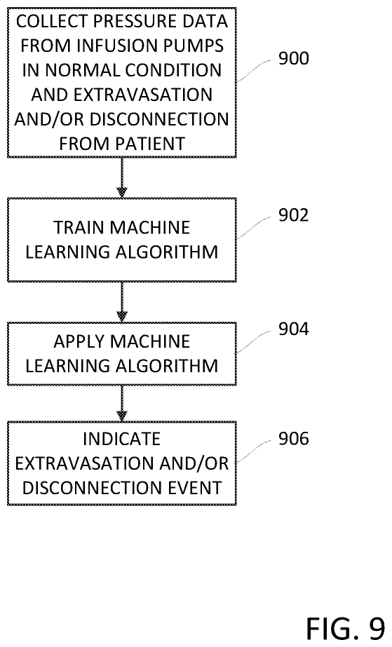

Referring now to FIG. 9, a system and method for using machine learning to predict extravasation (including infiltration) and/or disconnection of tubing from a patient will be described according to an illustrative embodiment. Extravasation refers to the leakage of an infusate out of the vein and into surrounding tissue. Leakage of a vesicant drug may cause tissue damage through blistering and/or ulceration. Extravasation may occur if various circumstances, such as if the administration of the infusate is too quick, the medication is very acidic or basic, or if there is an obstruction in the intravenous delivery line.

Disconnection of tubing from a patient occurs after completion of an infusion, but also may be unintended by a clinician, such as when a patient becomes confused and removes an infusion line from the infusion site. Disconnection may also be in advertent, such as when a line is stepped on or caught on another person or device. Extravasation and disconnection may trigger alerts so that the condition can be remedied in the clinical setting.

Extravasation and/or disconnection may be detected using any of the techniques, features, steps or algorithms described hereinabove with reference to FIGS. 7 and 8 (occlusion detection). As shown in FIG. 9, in this case the collected data again may comprise pressure data as a primary signal of extravasation and/or disconnection, which may be downstream pressure readings from an occlusion sensor. The detection target in this case may comprise extravasation and/or disconnection instead of (or in addition to) occlusion.

At block 900, a corpus of data may be collected from a plurality of infusion pumps. The data may comprise various types of infusion data and/or patient data described herein. The corpus of data may comprise pressure in the delivery lines used by the infusion pumps over time for a plurality of infusion conditions comprising normal operation and extravasation and/or disconnection of tubing from a patient. As examples, the data may comprise one or more of (or two or three or more of) drug name and/or viscosity, flow rate, infusion set type, infusion route, etc.

In some embodiments, the plurality of infusion pumps may capture running log files of downstream pressure data into a repository of data sets that may or may not be used to train a machine learning algorithm to identify extravasation and/or disconnection of a patient from a tubing set. When a clinician notes that an extravasation event or disconnection event has occurred, the clinician may manually input such an indication to the infusion pump, such that the data associated with the event is manually annotated or labelled. In some examples, the infusion pump may prompt an operator on the display screen and/or with an audible prompt to annotate infusion data associated with an event that occurred on the infusion pump. The prompts displayed may comprise two or more of a normal condition, extravasation, a disconnection from the patient, an occlusion, a transport condition, a bolus condition, etc. The infusion pump may then upload the infusion data and the indication of the event to a database (e.g., locally to a removable memory device, wirelessly to a server or cloud storage resource, etc.) for collection.

In some embodiments, the infusion pump may operate an algorithm configured to detect extravasation and/or disconnection of tubing automatically (i.e., without requiring human input) or with human confirmation of a an automatically detected condition.

At a block 902, the method may comprise training a machine learning algorithm using the corpus of data collected in block 900, the machine learning algorithm being trained to predict an occurrence of extravasation and/or disconnection of tubing from a patient.

At a block 904, the method may comprise using the machine learning algorithm to receive real time pressure data from an operating infusion pump and to predict an occurrence of extravasation and/or disconnection of tubing from a patient. For example, the machine learning algorithm may look at the real time pressure data and/or other infusion data input to the algorithm and detect an occurrence of an extravasation event and/or a disconnection of tubing from a patient (and/or an occlusion event, a bolus delivery event, a transport condition or event, etc.).

At a block 906, the method may comprise providing an indication of the extravasation and/or disconnection of tubing from a patient to a display on the operating infusion pump. The indication may comprise an instruction to pause the infusion. The method may further comprise automatically stopping and/or pausing the infusion in response to the detected extravasation event. The method may comprise continuing infusion but prompting an operator on the display that a potential extravasation event has been detected, such that the operator may take action by confirming to the pump the presence of the extravasation event and/or indicating no extravasation is present. This operator input and associated infusion data may be used as feedback data to further improve the machine learning algorithm.

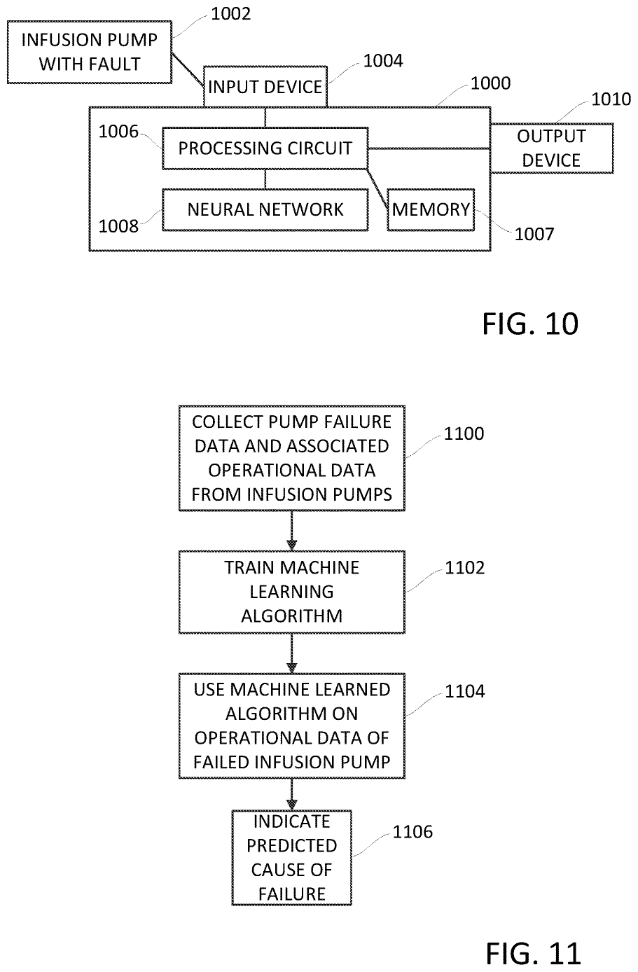

Referring now to FIGS. 10 and 11, a system and method of using machine learning to troubleshoot defects or failures in infusion pumps will be described, according to illustrative embodiments. Infusion pumps experiencing a failure of a component after manufacturing or assembly or while in use in a clinical setting may be diagnosed by a technician or biomedical engineer to identify the failure and repair the infusion pump. The cause of the failure may be difficult to isolate. In the embodiments described herein, infusion pump operational data, such as log file data stored within the infusion pump, may be used with a machine learned neural network to assist a technician in identifying the cause of the failure and optionally to suggest a repair.

FIG. 10 shows a computer system 1000 for predicting a cause of a failure in an infusion pump 1002. A data input device 1004 may be configured to receive a log file or other data file comprising infusion pump operational data for the infusion pump 1002. The data input device 1004 may comprise an input/output port, a Universal Serial Bus port, an RS-232 port, or a wired or wireless network connection configured to receive the log file from pump 1002 or from another computer. A processing circuit 1006 may be configured to retrieve the log file using data input device 1004 and to store the log file in a memory device 1007 (e.g., a local memory, such as RAM, EEPROM, flash memory, a solid-state drive, etc.).

The operational data or log file may comprise various types of data relating to the infusion pump. In one example, the log file comprises data generated during a pre-deployment manufacturer's test protocol (e.g., final acceptance test before shipping). After manufacture, but before shipping to a customer for use in a clinical setting, the infusion pump may run a self-test or an externally-run test protocol to check the operations of the infusion pump, such as proper functioning of the pump, sensors, display, input devices, power supply, etc. The infusion pump may be configured to store this data in a log file. The log file can then be run through computer system 1000 and neural network 1008 to help identify any failures introduced during manufacturing and/or assembly.

In another example, the log file may comprise data generated during use of the infusion pump in a clinical setting. While in use, infusion pump may self-detect and indicate a failure. A log file may be running throughout operation and/or be triggered to store data upon detection of the failure. A technician may then take the pump to computer 1000 and use neural network 1008 to help diagnose the cause of the failure.

A machine-learned neural network 1008 may be configured to receive the log file as input data and to process the log file to predict a cause of a failure in the infusion pump, neural network 1008 having been trained with log files associated with a variety of different failure events from this pump or other pumps. An output device 1010 may be configured to generate an indication of the cause of the failure of the infusion pump for display on a display device. Output device 1010 may be a display configured to display a message to a technician such as “pressure sensor A2 failed,” “door does not latch,” “pump failed,” etc. In some embodiments, output device 1010 may further be configured to identify a component to repair the infusion pump and to provide an indication of the identified component to replace to the display device. Output device 1010 may display “replace pressure sensor A2,” or “replace pump with part A123.” In further embodiments, output device 1010 may provide step-by-step instructions (e.g., at least two in sequence, at least three in sequence, etc.) instructing the operator how to repair the infusion pump to address the failure mode that was identified by the neural network. Output device 1010 may be configured to display: “Step 1: remove back cover of pump,” followed by subsequent steps that may be brought to the screen in response to a user indicating completion of a prior step.

In some embodiments, neural network 1008 may make a prediction which, after further analysis by a technician, is incorrect. In this case, the technician may annotate the log file with the correct cause of failure and submit the annotated log file as additional training data. The additional training data can be collated with other errant predictions from other infusion pumps and be used as feedback to further train neural network 1008. The updated neural network may then be redeployed (e.g., as a software update, etc.) for more robust diagnostic capabilities.

In some embodiments, the system and method of FIGS. 10-11 can be used to guide a technician in resolving defects introduced during assembly of infusion pump 1002. Some embodiments may provide predictive assessment of pump operation during assembly.

Referring now to FIG. 11, a method of using machine learning to diagnose a cause of an infusion pump failure in a healthcare setting will be described, according to illustrative embodiments. The method may alternatively be implemented as a computer system comprising a processing circuit configured to perform the steps. At a block 1100, the method comprises collecting a corpus of data comprising causes of infusion pump failure and associated operational data from infusion pumps. The method may comprise storing in a database all or many log files for a given pump, or for a given type of pump (syringe pumps, volume pumps, etc.) or model of pump (etc. Agilia Connect Syringe Pump by Fresenius Kabi). The operational data may be annotated with a failure root cause determined by a technician doing repairs. The operational data or log files may comprise different types of infusion pump data, such as infusion pump operation data (e.g., pressure in line that is sensed) or infusion pump programming data, such as a flow rate of medicament delivered by the infusion pump. Some of the infusion pump data may represent a signature for a given failure mode. For example, an infusion line pressure signal over time that shows a pressure during an infusion and then suddenly drops to zero while the infusion is still in progress could be a signature indicating a pressure sensor failure. Thus, the log file may include data indicating line pressure over, pump status at the time of the pressure drop (e.g., infusion, stopped, power loss, etc.), and/or other infusion data such as alerts that were triggered, the type of alert, etc. The causes of infusion pump failure may comprise a faulty electrical connection and/or a defective electronic sensor, among many other causes. The operational data collected at block 1100 and used to train the machine learning algorithm in block 1102 may comprise pressure sensor data, battery data, infusion pump programming data, and/or many other types of data relating to infusions performed by an infusion pump.

At a block 1102, the method may comprise training a machine learning algorithm using the corpus of data, the machine learning algorithm being trained to identify a possible cause of an infusion pump failure. Any of a number of machine learning training algorithms may be used, such as a deep learning, nearest neighbor, reinforcement learning, etc.

At a block 1104, the method may comprise using the machine learned algorithm to receive operational data from an infusion pump that has experienced a failure in the healthcare setting and to output a predicted cause of the infusion pump failure. This can be the same as one of the infusion pumps from which training data was collected in block 1100 or a different infusion pump. The infusion pump that has experienced a failure may have kept a running log file over time of infusion pump data and/or it may have stored certain infusion pump data or extra infusion pump data beyond that collected during normal operation in response to a detected fault condition of the pump. In some embodiments, the machine learning algorithm may be trained to identify the predetermined failure mode based on the received log file having a second signature substantially the same as a first signature received during training, as described above.

At a block 1106, the method may comprise providing an indication of the predicted cause of the infusion pump failure to a display device.

The method may comprise further features, such as manually diagnosing an infusion pump failure to identify a cause of a failure of the infusion pump and annotating a log file of the infusion pump with the identified cause of the failure. This annotated log file may be stored with the corpus of data collected in block 1100 and used to train the machine learning algorithm in block 1102.

In some embodiments, a feedback method may be provided to further improve the ability of the neural network to predict a cause of infusion pump failure. The method may further comprise manually diagnosing the infusion pump failure to identify a second cause of the failure different than the cause of the failure identified by the machine learned algorithm and annotating the log file of the infusion pump with the second cause of the failure. The machine learned algorithm may be retrained with the log file annotated with the second cause of the failure and then redeployed for more accurate predictions.

In some embodiments, one or more steps of FIG. 11 may be performed to identify causes of infusion pump failures introduced during manufacture and/or assembly. The collection and training may occur based on data collected from pumps after manufacture and/or assembly and before shipping to a customer. This may be a different trained machine learned algorithm than that used for diagnosing failure modes in the field. Some failures of pumps used in the field may relate to wear on components due to use over time. A machine learned algorithm that is tuned to the types of failures encountered by pumps in the field may be better at predicting the true cause of failures than one trained on both pre-clinical use pump failures and clinical use pump failures.

Infusion pumps have maintenance needs, some routine and some more emergent. Exemplary maintenance needs include the need to examine parts for wear and tear, the need for software updates, the need to replace batteries whether rechargeable or non-rechargeable, the need to confirm that lights, indicators and displays are working, calibration of the machine for accurate flow rate, running any embedded self-test algorithms, lubrication of parts such as a lead screw for a drive shaft of a syringe pump, and/or other maintenance needs. A machine learned neural network can predict the need for certain maintenance needs based on infusion pump data stored in a log file. The prediction can augment a maintenance need checklist based solely on time (hours or dates) in service. Simply relying on weekly, monthly, or annual maintenance checklists risks over-maintenance or under-maintenance of an infusion pump. A machine learned neural network system and method can provide a more intelligent approach.

In one embodiment, an infusion pump may comprise a processing circuit, a machine-learned neural network and an output device. The processing circuit may be configured to drive a motor to infuse a substance from a source through an infusion line to a patient. The processing circuit may be configured to store infusion data relating to the infusion. A wide range of infusion data can be stored and can be useful to the machine-learned neural network in predicting a need for a maintenance services. The infusion data may comprise data from any on-board sensor, vibration sensor, acceleration sensor, current or voltage sensor, microphone to capture noise levels which may indicate gear wear, pressure sensor data, infusion programming data (e.g., flow rate, drug name, therapy), care practice area (e.g., NICU, med/surg, ICU, etc.), hours of operation since deployment, duration of infusion, other infusion data report elements such as amount infused vs. programmed amount to infuse, battery life, battery voltage, number of pump stalls, frequency of pump stalls, etc.

A machine-learned neural network may be configured to receive the infusion data as input data and to process the infusion data to predict a need for a maintenance service for the infusion pump. For example, an increase in noise output may indicate gear wear and a need to maintain gears (e.g., lubricate, replace, etc.). As another example, an increase in power draw by a motor or other components may indicate a need to maintain moving components such as gears, etc. As another example, a drift or divergence between outputs of a plurality of sensors monitoring the same condition (e.g., pressure) may indicate a need to replace a pressure sensor. As another example, a counter tracking a number of disposable sets inserted into the device can be used to estimate total use of the infusion pump and therefore when a need for maintenance is likely to arise. A counter tracking a number of power on/off cycles may similarly be used, as may a counter counting a number of button presses.

An output device may be configured to generate an audible and/or visible alert in response to the predicted need for a maintenance service for the infusion pump. The alert may be provided on a display and/or speaker of the infusion pump itself having the need for maintenance so that a clinician can be notified that a technician may be needed to address the maintenance concern.

In some embodiments, the predicted need for maintenance may comprise a need to replace a motor. In some embodiments, the predicted need for maintenance may comprise a need to lubricate an actuator of a syringe pump. In some embodiments, the infusion data may comprise an indication of a frequency of pump stalls.

In some embodiments, the predicted need for maintenance may comprise replacing a rechargeable battery. In some embodiments, the infusion data may comprise an indication of battery voltage. In some embodiments, the predicted need for maintenance may be a need to maintain a pneumatic system of an infusion pump.

In some embodiments, the predicted need for maintenance comprises a need to calibrate the motor. In some embodiments, the predicted need for maintenance comprises a need to run an infusion pump self-test algorithm embedded in firmware on the infusion pump.

In some embodiments, a feedback method may be provided to further improve the ability of the neural network to predict a need for maintenance. The method may further comprise manually identifying a need for a second maintenance of the infusion pump different than a first maintenance identified by the machine learned algorithm and annotating operational data of the infusion pump with the second maintenance need. The machine learned algorithm may be retrained with the annotated data with the second maintenance need and then redeployed for more accurate predictions.

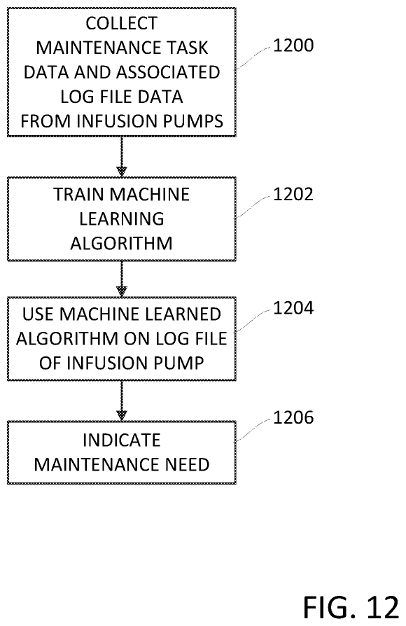

Referring to FIG. 12, a method of using machine learning to predict a maintenance need of an infusion pump will be described according to illustrative embodiments. At a block 1200, the method may comprise collecting a corpus of data comprising maintenance tasks of infusion pumps and associated log file data from the infusion pumps. The association between the log file data and the maintenance tasks may be done by manually annotating the log file data with associated maintenance needs. For example, a technician may monitor a pump and make a determination that the pump has a maintenance need. The technician may then acquire log data from the infusion pump and label or annotate the data with a maintenance need indication. The log data may comprise any of a variety of infusion pump data, and in some embodiments may comprise at least two (or at least three) of pump operating time, pump stalls, battery voltage, alerts, etc.

At a block 1202, the method may comprise training a machine learning algorithm using the corpus of data, the machine learning algorithm being trained to predict a maintenance need of an infusion pump. At a block 1204, the method may comprise using the machine learning algorithm to receive a log file from an infusion pump and to output a maintenance need of the infusion pump.

At a block 1206, the method may comprise providing an indication of the maintenance need of the infusion pump to a display device.

### OTHER ASPECTS

### Aspect 1

A computer system, comprising:
a data aggregator configured to receive infusion pump programming data comprising one or more of dose limit, dose, dose rate, rate, volume, duration, administration site and diagnosis from medical infusion pumps in use at a plurality of different care facilities;
a machine-learned neural network configured to receive the infusion pump programming data and to process the infusion pump programming data to identify a set of recommended dose limit settings;
a storage unit configured to store the set of recommended dose limit settings; and
a reporting unit configured to transmit the set of recommended dose limit settings in response to a received request.

### Aspect 2

The computer system of Aspect 1, further comprising a programming unit configured to program a set of medical infusion pumps based on the set of recommended dose limit settings, wherein the medical infusion pumps are programmed to infuse medications based on the set of recommended dose limit settings.

### Aspect 3

The computer system of Aspect 1 or 2, wherein the infusion pump programming data comprises hard limits and soft limits used by the medical infusion pumps and care practice areas within the care facilities associated with each of the hard limits and soft limits.

### Aspect 4

The computer system of Aspect 3, wherein the care practice areas comprise intensive care unit, operating room, or pediatric unit.

### Aspect 5

The computer system of any one of Aspects 1 to 4, wherein the data aggregator is configured to aggregate the infusion pump programming data from medical infusion pumps in use at different corporate entities.

### Aspect 6

The computer system of any one of Aspects 1 to 5, further comprising a drug library editor module configured to receive user inputs for dose limits and display selected dose limits, wherein the set of recommended dose limit settings are displayed within the drug library editor.

### Aspect 7

The computer system of any one of Aspects 1 to 6, wherein the infusion pump programming data further comprises patient population data comprising pediatric, adult and geriatric.

### Aspect 8

The computer system of any one of Aspects 1 to 7, wherein the infusion pump programming data further comprises pump alert data associated with each of a plurality of dose limits.

### Aspect 9

A method of identifying a potential adverse event associated with drug delivery parameters based on patient physiological data, comprising:
receiving patient physiological data;
receiving a drug delivery parameter for a proposed drug therapy to be administered to the patient;
processing the patient physiological data and the drug delivery parameter with a machine learned neural network, wherein the machine learned neural network identifies a potential adverse event associated with delivery of the proposed drug therapy to the patient;
generating an alert message based on the identified potential adverse event;
transmitting the alert message to an infusion pump; and
generating an audible and/or visual alert at the infusion pump to alert an operator to the existence of a potential adverse event.

### Aspect 10

The method of Aspect 9, wherein the patient physiological data comprises a plurality of patient vital signs.

### Aspect 11

The method of Aspect 9 or 10, wherein the patient physiological data comprises clinical laboratory data including a result of at least one blood test.

### Aspect 12

The method of any one of Aspects 9 to 11, wherein the patient physiological data comprises at least one of a patient diagnosis, patient age and patient acuity data.

### Aspect 13

The method of any one of Aspects 9 to 12, wherein the drug delivery parameter for the proposed drug therapy comprises a drug name and a dosage.

### Aspect 14

The method of any one of Aspects 9 to 13, further comprising:
using the machine learned neural network to generate a recommended clinical action to take in view of the potential adverse event; and
transmitting the recommended clinical action to the infusion pump; and
displaying an indication of the recommended clinical action on a display of the infusion pump.

### Aspect 15

The method of Aspect 14, further comprising:
transmitting an indication of one or more of the drug delivery parameters associated with the indication of the potential adverse event; and
displaying the indication of the one or more of the drug delivery parameters on the display of the infusion pump.

### Aspect 16

A method of using machine learning to avoid adverse events when administering a drug therapy to a patient, comprising:
collecting a corpus of data comprising potential adverse events associated with patient physiological data and drug delivery parameters;
training a machine learning algorithm using the corpus of data, the machine learning algorithm being trained to identify a possible adverse event should a proposed drug therapy be administered to a patient having certain patient physiological data;
using the machine learning algorithm to receive a proposed drug therapy and patient physiological data and to identify the possible adverse event; and
providing an indication of the possible adverse event to a display device.

### Aspect 17

The method of Aspect 16, wherein the corpus of data is created based at least in part on medical journal research.

### Aspect 18

The method of Aspect 16 or 17, wherein the possible adverse event comprises an inadvertent bleed when the proposed drug therapy comprises heparin.

### Aspect 19

The method of any one of Aspects 16 to 18, further comprising using the machine learning algorithm to recommend a modification to at least one parameter of the proposed drug therapy.

### Aspect 20

The method of Aspect 19, further comprising using the machine learning algorithm to generate a recommended clinical action should the proposed drug therapy be administered to the patient.

### Aspect 21

The method of Aspect 20, further comprising transmitting the recommended modification and/or the recommended clinical action to an infusion pump, wherein the infusion pump displays the modification and/or the recommended clinical action on a display integrated into the infusion pump.

### Aspect 22

The method of Aspect 21, wherein the received patient physiological data comprises the patient's activated partial thromboplastin time, wherein the indication of a possible adverse event comprises an indication of an inadvertent bleed.

### Aspect 23

The method of Aspect 22, wherein the recommended clinical action comprises a recommendation to test the patient's activated partial thromboplastin time.

### Aspect 24

The method of Aspect 23, wherein the recommended modification comprises a lower dose of the proposed drug therapy.

### Aspect 25

A method of using machine learning to identify misuse of controlled medications, comprising:
collecting a corpus of data comprising first infusion data and misuse data indicating misuse of a controlled medication;
training a machine learning algorithm using the corpus of data, the machine learning algorithm being trained to identify a possible misuse event based on second infusion pump data;
using the machine learning algorithm to receive the second infusion pump data to identify the possible misuse event; and
providing an indication of the possible misuse event to a display device.

### Aspect 26

The method of Aspect 25, wherein the controlled medication comprises a drug classified as Schedule II under the Controlled Substances Act.

### Aspect 27

The method of Aspect 26, wherein the controlled medication comprises oxycodone.

### Aspect 28

The method of any one of Aspects 25 to 27, wherein the corpus of data is collected from multiple different healthcare facilities in different geographic locations.

### Aspect 29

The method of any one of Aspects 25 to 28, wherein the infusion data comprises an indication whether a medication infused is a narcotic.

### Aspect 30

The method of any one of Aspects 25 to 29, wherein the identified possible misuse event comprises underinfusion of controlled medications relative to non-controlled medications.

### Aspect 31

The method of Aspect 30, wherein the identified possible misuses event comprises underinfusion of controlled medications relative to non-controlled medications by a particular staff member.

### Aspect 32

The method of any one of Aspects 25 to 31, wherein the first infusion data is manually annotated to provide the misuse data indicating the medicament has been misused.

### Aspect 33

A computer system, comprising:
a data aggregator configured to receive infusion data from medical infusion pumps in use to dispense controlled medications;
a machine-learned neural network configured to receive the infusion data and to identify potential misuse of one of the controlled medications;
a storage unit configured to store identified misuse of the controlled medication; and
a reporting unit configured to transmit an alert regarding the identified misuse of the controlled medication to a destination device.

### Aspect 34

The computer system of Aspect 33, wherein the controlled medications being dispensed comprise Schedule II medications and the identified potential misuse is potential misuse of a Schedule II medication.

### Aspect 35

The computer system of Aspect 34, wherein the reporting unit is configured to transmit the alert using a notification server to care unit leadership and/or a designated hospital monitoring personnel.

### Aspect 36

The computer system of any one of Aspects 33 to 35, wherein the machine-learned neural network was trained using one or more of a convolutional neural network, deep learning, Bayesian network, nearest neighbor, or reinforcement learning.

### Aspect 37

The computer system of any one of Aspects 33 to 36, wherein the machine-learned neural network is configured to identify underinfusion of one of the controlled medications by a particular clinician.

### Aspect 38

The computer system of any one of Aspects 33 to 37, wherein the machine-learned neural network is configured to identify underinfusion of one of the controlled medications by a care practice area.

### Aspect 39

The computer system of any one of Aspects 33 to 38, wherein the reporting unit is configured to transmit the alert as an advisory to a plurality of different healthcare facilities.

### Aspect 40

The computer system of any one of Aspects 33 to 39, further comprising receiving feedback in response to the alert and retraining the machine-learned neural network based on the feedback.

### Aspect 41

The computer system of any one of Aspects 33 to 40, wherein the machine-learned neural network is configured to identify a pattern of infusion data as indicating misuse for a first care practice area and to identify the same pattern of infusion data as not indicating misuse for a second care practice area.

### Aspect 42

An infusion pump, comprising:
a processing circuit configured to drive an actuator to infuse a substance from a source to a patient, the processing circuit configured to store infusion data relating to the infusion;
a machine-learned neural network configured to receive the infusion data as input data and to process the infusion data to predict an occurrence of an occlusion in the line; and
an output device configured to generate an audible and/or visible alert in response to the predicted occurrence of an occlusion in the line.

### Aspect 43

The infusion pump of Aspect 42, wherein the infusion data comprises data from a sensor configured to determine pressure in the infusion line, wherein the machine-learned neural network is configured to process the sensor data to predict the occurrence of an occlusion in the line.

### Aspect 44

The infusion pump of Aspect 43, wherein the sensor comprises a motor current sensor and/or a force sensor.

### Aspect 45

The infusion pump of Aspect 43, wherein the sensor is configured to determine downstream pressure in the line between a pumping actuator and the patient.

### Aspect 46

The infusion pump of any one of Aspects 42 to 45, wherein the infusion data comprises a characteristic of the infusion line, wherein the machine-learned neural network is configured to receive the characteristic of the infusion line as input data and to process the characteristic of the infusion line to predict the occurrence of an occlusion in the line.

### Aspect 47

The infusion pump of Aspect 46, wherein the characteristic of the infusion line is a bore size of the infusion line.

### Aspect 48

The infusion pump of Aspect 46, wherein the infusion data further comprises a viscosity of the substance being infused, wherein the machine-learned neural network is configured to receive the viscosity of the substance being infused as input data and to process the viscosity of the substance being infused to predict the occurrence of an occlusion in the line.

### Aspect 49

The infusion pump of Aspect 48, wherein the infusion data further comprises downstream pressure in the infusion line, wherein the machine-learned neural network is configured to receive the downstream pressure in the infusion line as input data and to process the downstream pressure in the infusion line to predict the occurrence of an occlusion in the line.

### Aspect 50

A method of using machine learning to predict an occurrence of an occlusion in a line of a delivery set driven by an infusion pump, comprising:
collecting a corpus of data from a plurality of infusion pumps, wherein the corpus of data comprises pressure in delivery lines used by the infusion pumps over time for a plurality of infusion conditions comprising normal operation and an occlusion condition;
training a machine learning algorithm using the corpus of data, the machine learning algorithm being trained to predict an occurrence of an occlusion in a line; using the machine learning algorithm to receive real time pressure data from an operating infusion pump and to predict an occurrence of an occlusion in the operating infusion pump; and
providing an indication of the occlusion to a display on the operating infusion pump.

### Aspect 51

The method of Aspect 50, wherein the corpus of data is collected from infusion pumps operating at different healthcare facilities by different corporate entities.

### Aspect 52

The method of Aspect 50 or 51, wherein the corpus of data further comprises pressure in delivery lines over time for a bolus condition.

### Aspect 53

The method of any one of Aspects 50 to 52, wherein the corpus of data further comprises pressure in delivery lines over time for a transport condition indicating the infusion pump was being transported from one clinical location to another clinical location.

### Aspect 54

The method of any one of Aspects 50 to 53, wherein the pressure in delivery lines used by the infusion pumps over time comprises a signature of an occlusion.

### Aspect 55

The method of any one of Aspects 50 to 54, wherein the corpus of data comprises two or more of drug name, drug viscosity, programmed infusion rate, and administration set type, wherein the machine language algorithm is trained with the two or more of drug name, drug viscosity, programmed infusion rate, and administration set type.

### Aspect 56

The method of any one of Aspects 50 to 55, wherein the corpus of data comprises two or more of patient weight, infusion route and care practice area, wherein the machine language algorithm is trained with the two or more of patient weight, infusion route and care practice area.

### Aspect 57

The method of any one of Aspects 50 to 56, further comprising operating the plurality of infusion pumps with an occlusion detection algorithm and recording pressure in delivery lines used by the infusion pumps over time in response to the infusion pump detecting an occlusion condition, further comprising training the machine learning algorithm using the recorded pressure over time.

### Aspect 58

The method of any one of Aspects 50 to 57, wherein the operating infusion pump is configured to prompt an operator to confirm a presence of the occlusion, wherein infusion data from the pump is annotated to indicate the presence of or an absence of the occlusion condition, wherein the annotated infusion data is used as feedback data to further train the machine learned neural network.

### Aspect 59

An infusion pump, comprising:
a processing circuit configured to drive an actuator to infuse a substance to a patient, the processing circuit configured to store infusion data relating to the infusion;
a machine-learned neural network configured to receive the infusion data as input data and to process the infusion data to predict an occurrence of extravasation and/or disconnection of tubing from a patient; and
an output device configured to generate an audible and/or visible alert in response to the predicted occurrence of extravasation and/or disconnection of tubing from a patient.

### Aspect 60

The infusion pump of Aspect 59, wherein the infusion data comprises data from a sensor configured to determine pressure in the infusion line, wherein the machine-learned neural network is configured to process the sensor data to predict the occurrence of extravasation and/or disconnection of tubing from a patient.

### Aspect 61

The infusion pump of Aspect 60, wherein the sensor comprises a force sensor.

### Aspect 62

The infusion pump of Aspect 60, wherein the sensor is configured to determine downstream pressure in the line between a pumping actuator and the patient.

### Aspect 63

The infusion pump of any one of Aspects 59 to 62, wherein the infusion data comprises a characteristic of the infusion line, wherein the machine-learned neural network is configured to receive the characteristic of the infusion line as input data and to process the characteristic of the infusion line to predict the occurrence of extravasation and/or disconnection of tubing from a patient.

### Aspect 64

The infusion pump of Aspect 63, wherein the characteristic of the infusion line is a bore size of the infusion line.

### Aspect 65

The infusion pump of Aspect 63, wherein the infusion data further comprises a viscosity of the substance being infused, wherein the machine-learned neural network is configured to receive the viscosity of the substance being infused as input data and to process the viscosity of the substance being infused to predict the occurrence of extravasation and/or disconnection of tubing from a patient.

### Aspect 66

The infusion pump of Aspect 65, wherein the infusion data further comprises downstream pressure in the infusion line, wherein the machine-learned neural network is configured to receive the downstream pressure in the infusion line as input data and to process the downstream pressure in the infusion line to predict the occurrence of extravasation and/or disconnection of tubing from a patient.

### Aspect 67

A method of using machine learning to predict an occurrence of extravasation and/or disconnection of tubing from a patient receiving therapy from an infusion pump, comprising:
collecting a corpus of data from a plurality of infusion pumps, wherein the corpus of data comprises pressure in delivery lines used by the infusion pumps over time for a plurality of infusion conditions comprising normal operation and extravasation and/or disconnection of tubing from a patient;
training a machine learning algorithm using the corpus of data, the machine learning algorithm being trained to predict an occurrence of extravasation and/or disconnection of tubing from a patient;
using the machine learning algorithm to receive real time pressure data from an operating infusion pump and to predict an occurrence of extravasation and/or disconnection of tubing from a patient; and
providing an indication of the extravasation and/or disconnection of tubing from a patient to a display on the operating infusion pump.

### Aspect 68

The method of Aspect 67, wherein the corpus of data is collected from infusion pumps operating at different healthcare facilities by different corporate entities.

### Aspect 69

The method of Aspect 67 or 68, wherein the corpus of data further comprises pressure in delivery lines over time for a bolus condition.

### Aspect 70

The method of any one of Aspects 67 to 69, wherein the corpus of data further comprises pressure in delivery lines over time for a transport condition indicating the infusion pump was being transported from one clinical location to another clinical location.

### Aspect 71

The method of any one of Aspects 67 to 70, wherein the pressure in delivery lines used by the infusion pumps over time comprises a signature of extravasation and/or disconnection of tubing from a patient.

### Aspect 72

The method of any one of Aspects 67 to 71, wherein the corpus of data comprises two or more of drug name, drug viscosity, programmed infusion rate, and administration set type, wherein the machine language algorithm is trained with the two or more of drug name, drug viscosity, programmed infusion rate, and administration set type.

### Aspect 73

The method of any one of Aspects 67 to 72, wherein the corpus of data comprises two or more of patient weight, infusion route and care practice area, wherein the machine language algorithm is trained with the two or more of patient weight, infusion route and care practice area.

### Aspect 74

The method of any one of Aspects 67 to 73, wherein the corpus of data is annotated by a human operator to indicate the extravasation and/or disconnection from a patient.

### Aspect 75

The method of Aspect 74, wherein the plurality of infusion pumps are configured to prompt an operator to annotate infusion data associated with an event that occurred on the infusion pump.

### Aspect 76

The method of Aspect 75, wherein the prompts comprise two or more of a normal condition, extravasation, and a disconnection of tubing from the patient.

### Aspect 77

A method of using machine learning to diagnose a cause of an infusion pump failure in a healthcare setting, comprising:
collecting a corpus of data comprising causes of infusion pump failure and associated operational data from infusion pumps;
training a machine learning algorithm using the corpus of data, the machine learning algorithm being trained to identify a possible cause of an infusion pump failure;
using the machine learning algorithm to receive operational data from an infusion pump that has experienced a failure in the healthcare setting and to output a predicted cause of the infusion pump failure; and
providing an indication of the predicted cause of the infusion pump failure to a display device.

### Aspect 78

The method of Aspect 77, further comprising:
manually diagnosing an infusion pump failure to identify a cause of a failure of the infusion pump;
annotating a log file of the infusion pump with the identified cause of the failure; and
storing the annotated log file with the corpus of data used to train the machine learning algorithm.

### Aspect 79

The method of Aspect 77 or 78, further comprising:
identifying a component to repair the infusion pump; and
providing an indication of the identified component to replace to the display device.

### Aspect 80

The method of any one of Aspects 77 to 79, further comprising:
manually diagnosing the infusion pump failure to identify a cause of a failure of the infusion pump;
annotating the operational data of the infusion pump with the identified cause of the failure; and
storing the annotated operational data with the corpus of data used to train the machine learning algorithm.

### Aspect 81

The method of any one of Aspects 77 to 80, further comprising:
manually diagnosing the infusion pump failure to identify a second cause of the failure different than the identified cause of the failure;
annotating the operational data of the infusion pump with the second cause of the failure; and
retraining the machine learning algorithm with the annotated operational data.

### Aspect 82

The method of any one of Aspects 77 to 81, wherein the causes of the infusion pump failures are failures introduced during manufacture and/or assembly.

### Aspect 83

The method of any one of Aspects 77 to 82, wherein the causes of infusion pump failures comprise a faulty electrical connection and/or a defective electronic sensor.

### Aspect 84

The method of any one of Aspects 77 to 83, wherein the operational data used to train the machine learning algorithm comprise pressure sensor data, battery data, and/or infusion pump programming data.

### Aspect 85

The method of Aspect 84, wherein the operational data used to train the machine learning algorithm comprises infusion pump programming data comprising a flow rate of medicament delivered by the infusion pump.

### Aspect 86

The method of any one of Aspects 77 to 85, wherein the operational data used to train the machine learning algorithm represent a signature correlated to a predetermined failure mode, wherein the machine learning algorithm is trained to identify the predetermined failure mode based on the received operational data having a second signature substantially the same as the first signature.

### Aspect 87

A computer system for predicting a cause of a failure in an infusion pump, comprising:
a data input device configured to receive operational data for the infusion pump;
a processing circuit configured to retrieve the operational data using the data input device and to store the operational data in a memory device;
a machine-learned neural network configured to receive the operational data as input data and to process the operational data to predict a cause of a failure in the infusion pump; and
an output device configured to generate an indication of the cause of the failure of the infusion pump for display on a display device.

### Aspect 88

The computer system of Aspect 87, wherein the operational data comprises data generated during a pre-deployment manufacturer's test protocol.

### Aspect 89

The computer system of Aspect 87 or 88, wherein the operational data comprises data generated during use of the infusion pump in a clinical setting.

### Aspect 90

The computer system of any one of Aspects 87 to 89, wherein the processing circuit is configured to collect input data indicating the predicted cause of the failure is incorrect.

### Aspect 91

The computer system of Aspect 90, wherein the processing circuit is configured to collect input data derived from a manual diagnostic procedure indicating a correction to the incorrect predicted cause, wherein the processing circuit is configured to train the machine-learned neural network with the input data comprising the correction.

### Aspect 92

An infusion pump, comprising:
a processing circuit configured to drive an actuator to infuse a substance to a patient, the processing circuit configured to store infusion data relating to the infusion;
a machine-learned neural network configured to receive the infusion data as input data and to process the infusion data to predict a need for a maintenance service for the infusion pump; and
an output device configured to generate an audible and/or visible alert in response to the predicted need for a maintenance service for the infusion pump.

### Aspect 93

The infusion pump of Aspect 92, wherein the predicted need for maintenance comprises a need to replace a motor.

### Aspect 94

The infusion pump of Aspect 92 or 93, wherein the predicted need for maintenance comprises a need to lubricate an actuator of a syringe pump.

### Aspect 95

The infusion pump of Aspect 94, wherein the infusion data comprises an indication of a frequency of pump stalls.

### Aspect 96

The infusion pump of any one of Aspects 92 to 95, wherein the predicted need for maintenance comprises replacing a rechargeable battery.

### Aspect 97

The infusion pump of Aspect 96, wherein the infusion data comprises an indication of battery voltage.

### Aspect 98

The infusion pump of any one of Aspects 92 to 97 wherein the predicted need for maintenance comprises a need to calibrate the motor.

### Aspect 99

The infusion pump of any one of Aspects 92 to 98, wherein the predicted need for maintenance comprises a need to run an infusion pump self-test algorithm.

### Aspect 100

A method of using machine learning to predict a maintenance need of an infusion pump, comprising:
collecting a corpus of data comprising maintenance tasks of infusion pumps and associated log file data from the infusion pumps;
training a machine learning algorithm using the corpus of data, the machine learning algorithm being trained to predict a maintenance need of an infusion pump;
using the machine learning algorithm to receive a log file from an infusion pump and to output a maintenance need of the infusion pump; and
providing an indication of the maintenance need of the infusion pump to a display device.

### Aspect 101

The method of Aspect 100, further comprising manually annotating the log file data with associated maintenance needs.

### Aspect 102

The method of Aspect 100 or 101, wherein the log file data comprises at least two of pump operating time, pump stalls, battery voltage, and alerts.

The embodiments described herein are illustrative only. Although only a few embodiments of the present disclosure have been described in detail, those skilled in the art who review this disclosure will appreciate that many modifications are possible (e.g., variations in sizes, dimensions, structures, shapes and proportions of the various elements, values of parameters, mounting arrangements, etc.) without materially departing from the novel teachings and advantages of the subject matter recited herein. The described program components and systems can generally be integrated together in a single product or packaged into multiple products. Accordingly, all such modifications are intended to be included within the scope of the present disclosure as described herein. The order sequence of any process or method steps may be varied or re-sequenced according to alternative embodiments. Steps, blocks, or features of one embodiment may be combined with other embodiments to realize new patentable concepts. Other substitutions, modifications, changes, and/or omissions may be made in the design, operating conditions and arrangement of the preferred and other illustrative embodiments without departing from the scope of the present disclosure as expressed herein. Moreover, although features may be described herein as acting in certain combinations and even initially claimed as such, one or more features from a claimed combination can in some cases be excised from the combination, and the claimed combination may be directed to a subcombination or variation of a subcombination.

1. A computer system, comprising:
a data aggregator configured to receive infusion pump programming data comprising one or more of dose limit, dose, dose rate, rate, volume, duration, administration site and diagnosis from medical infusion pumps in use at a plurality of different care facilities;
a machine-learned neural network configured to receive the infusion pump programming data and to process the infusion pump programming data to identify a set of recommended dose limit settings;
a storage unit configured to store the set of recommended dose limit settings; and
a reporting unit configured to transmit the set of recommended dose limit settings in response to a received request.

2. The computer system of claim 1, further comprising a programming unit configured to program a set of medical infusion pumps based on the set of recommended dose limit settings, wherein the medical infusion pumps are programmed to infuse medications based on the set of recommended dose limit settings.

3. The computer system of claim 1, wherein the infusion pump programming data comprises hard limits and soft limits used by the medical infusion pumps and care practice areas within the care facilities associated with each of the hard limits and soft limits, wherein the care practice areas comprise intensive care unit, operating room, or pediatric unit.

4. (canceled)

5. The computer system of claim 1, wherein the data aggregator is configured to aggregate the infusion pump programming data from medical infusion pumps in use at different corporate entities.

6. The computer system of claim 1, further comprising a drug library editor module configured to receive user inputs for dose limits and display selected dose limits, wherein the set of recommended dose limit settings are displayed within the drug library editor.

7. The computer system of claim 1, wherein the infusion pump programming data further comprises patient population data comprising pediatric, adult and geriatric.

8. The computer system of claim 1, wherein the infusion pump programming data further comprises pump alert data associated with each of a plurality of dose limits.

9-32. (canceled)

33. The computer system of claim 1, wherein:
the data aggregator is configured to receive infusion data from medical infusion pumps in use to dispense controlled medications;
the machine-learned neural network is configured to receive the infusion data and to identify potential misuse of one of the controlled medications;
the storage unit is configured to store identified misuse of the one of the controlled medications; and
the reporting unit is configured to transmit an alert regarding the identified misuse of the one of the controlled medications to a destination device.

34. The computer system of claim 33, wherein the controlled medications being dispensed comprise Schedule II medications and the identified potential misuse is potential misuse of a Schedule II medication, and wherein the reporting unit is configured to transmit the alert using a notification server to care unit leadership and/or a designated hospital monitoring personnel.

35. (canceled)

36. The computer system of claim 1, wherein the machine-learned neural network was trained using one or more of a convolutional neural network, deep learning, Bayesian network, nearest neighbor, or reinforcement learning.

37. The computer system of claim 33, wherein the machine-learned neural network is configured to identify underinfusion of the one of the controlled medications by a particular clinician or by a care practice area.

38. (canceled)

39. The computer system of claim 33, wherein the reporting unit is configured to transmit the alert as an advisory to a plurality of different healthcare facilities.

40. The computer system of claim 33, further comprising receiving feedback in response to the alert and retraining the machine-learned neural network based on the feedback.

41. The computer system of claim 33, wherein the machine-learned neural network is configured to identify a pattern of infusion data as indicating misuse for a first care practice area and to identify the same pattern of infusion data as not indicating misuse for a second care practice area.

42-102. (canceled)

103. The computer system of claim 1, wherein:
the data aggregator is configured to receive patient physiological data and drug delivery parameters for proposed drug therapies to be administered;
the machine-learned neural network is configured to receive a patient physiological data and a drug delivery parameter for a proposed drug therapy to be administered and to identify a potential adverse event associated with the drug delivery parameter based on patient physiological data;
the storage unit is configured to store identified potential adverse event associated with the drug delivery parameter; and
the reporting unit is configured to transmit an alert message based on the identified potential adverse event to an infusion pump,
wherein the infusion pump generates an audible and/or visual alert to alert an operator to the existence of the identified potential adverse event.

104. The computer system of claim 103, wherein the patient physiological data comprises at least one of:
a plurality of patient vital signs;
clinical laboratory data including a result of at least one blood test;
a patient diagnosis;
patient age; and
patient acuity data.

105. The computer system of claim 103, wherein the drug delivery parameter for the proposed drug therapy comprises a drug name and a dosage.

106. The computer system of claim 103, wherein:
the machine-learned neural network is configured to generate a recommended clinical action to take in view of the potential adverse event; and
the reporting unit is configured to transmit the recommended clinical action to the infusion pump,
wherein the infusion pump is configured to display an indication of the recommended clinical action on a display of the infusion pump.

107. The computer system of claim 106, wherein:
the reporting unit is configured to transmit an indication of one or more of the drug delivery parameters associated with the indication of the potential adverse event,
wherein the infusion pump is configured to display an indication of the one or more of the drug delivery parameters on the display of the infusion pump

108. The computer system of claim 106, wherein:
the received patient physiological data comprises the patient's activated partial thromboplastin time,
the potential adverse event comprises an inadvertent bleed, and
the recommended clinical action comprises at least one of to test the patient's activated partial thromboplastin time and to lower a dose of the proposed drug therapy.
